(19) United States
(12) Patent Application Publication
(10) Pub. No.: US 2012/0232038 A1
(43) Pub. Date: Sep. 13, 2012

(54) CRYSTALLINE FORMS OF (3R, 3AS, 6AR) - HEXAHYDROFURO [2,3-B] FURAN-3-YL (1S,2R) - (1-{4-[ (DIETHOXYPHOSPHORYL) METHOXY] PHENY1}-3-HYDROXY-4- [4-METHOXY-N- (2-METHYLPROPYL) BENZENESUL - FONAMIDO] BUTAN-2-YL) CARBAMATE

(75) Inventors: Ernest Anthony Carra, Foster City, CA (US); Anna Chiu, Mountain View, CA (US); Jesper Alexis Jernelius, San Francisco, CA (US); Fang Wang, Foster City, CA (US)

(73) Assignee: Gilead Sciences, Inc., Foster City, CA (US)

(21) Appl. No.: 13/378,250

(22) PCT Filed: Jun. 17, 2010
(86) PCT No.: PCT/US10/38989

§ 371 Date: May 30, 2012

Related U.S. Application Data
(60) Provisional application No. 61/220,500, filed on Jun. 25, 2009.

(51) Int. Cl. A61K 31/665; A61P 31/18; C07F 9/40

(52) U.S. Cl. 514/100; 549/220

(57) ABSTRACT

The present inventions provides crystalline forms of (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate, methods of preparing the crystalline forms, pharmaceutical compositions containing the crystalline forms, and therapeutic uses thereof.

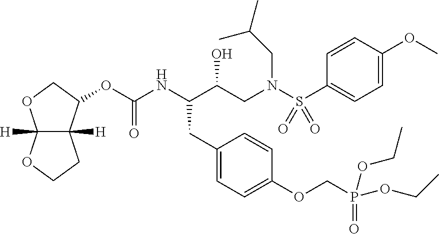

### FIELD OF THE INVENTION

The present invention relates to novel crystalline forms of (3R,3aS,6aR)-hexahydrofuro[2,3-b]furan-3-yl(1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate, methods of preparing the crystalline forms, pharmaceutical compositions comprising the novel crystalline forms, and methods of using the crystalline forms and the pharmaceutical compositions for treating diseases or conditions.

### BACKGROUND OF THE INVENTION

(3R,3aS,6aR)-Hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate (Compound I), represented by the structure below

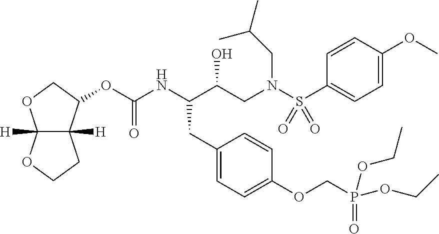

is a bis-tetrahydrofuran-based peptidomimetic HIV protease inhibitor (PI) for treating HIV infection. This structure is also known as Compound I. Compound I, its functionality, methods of making and therapeutic uses are described in U.S. Pat. No. 7,649,015B2 issued on Jan. 19, 2010. Compound I acts to inhibit retroviral proteases and thus inhibits the viral replication. Accordingly, Compound I is useful for treating human patients infected with a human retrovirus, such as human immunodeficiency virus (strains HIV-1 or HIV-2), or human T-cell leukemia viruses (HTLV-1 or HTLV-2) which results in acquired immunodeficiency syndrome (AIDS) and related diseases, etc. Currently Compound I is known to exist in the amorphous form. The amorphous form is sticky, difficult to disperse and prone to deliquescence. Thus, the amorphous form of Compound I is challenging for drug purification, isolation, handling, processing and formulation. Many efforts have been made to prepare a different form that is more desirable for drug handling, processing and formulation. However, it has been very difficult to generate a crystalline form of Compound I. The inventors of the present invention have surprisingly discovered that Compound I can be prepared as a novel crystalline polymorph named Form I, which is more stable and easy to handle in processing.

### SUMMARY OF THE INVENTION

The present invention provides novel crystalline forms of (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate (Compound I) represented by the structure below,

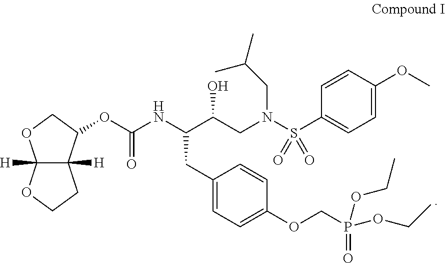

Non-limiting examples include for example, crystalline form I (Form I) and co-crystal forms of Compound I comprising Compound I and various co-formers.

In one aspect, the present invention provides a novel crystalline form of Compound I named as Form I.

In another aspect, the present invention provides co-crystals of Compound I comprising Compound I and a co-crystal former. In one embodiment, the co-crystal former is selected from L-tartaric acid, D-tartaric acid, malonic acid, L-malic acid, D-malic acid or benzoic acid, and the like.

In another aspect, the present invention provides methods for preparing the crystalline forms of the present invention.

In another aspect, the present invention provides methods of using the crystalline forms or the pharmaceutical composition containing the crystalline forms of the present invention for therapeutic applications.

### BRIEF DESCRIPTION OF THE FIGURES

FIG. 1 shows X-ray powder diffraction (XRPD) patterns of Compound I crystalline Form I and amorphous form.

FIG. 2 shows differential scanning calorimetry (DSC) of Compound I crystalline Form I.

FIG. 3 shows thermogravimetric analysis of Compound I crystalline Form I.

FIG. 4 shows dynamic vapor sorption of Compound I crystalline Form I.

FIG. 5 shows moisture uptake of Compound I amorphous material.

FIG. 6 shows comparison of XRPDs of 2 week sample of Compound I slurried in water with reference to Compound I Form I.

FIG. 7 shows X-ray powder diffraction (XRPD) patterns of Compound I/L-tartaric acid co-crystal Form A.

FIG. 8 shows differential scanning calorimetry (DSC) of Compound I/L-tartaric acid co-crystal Form A.

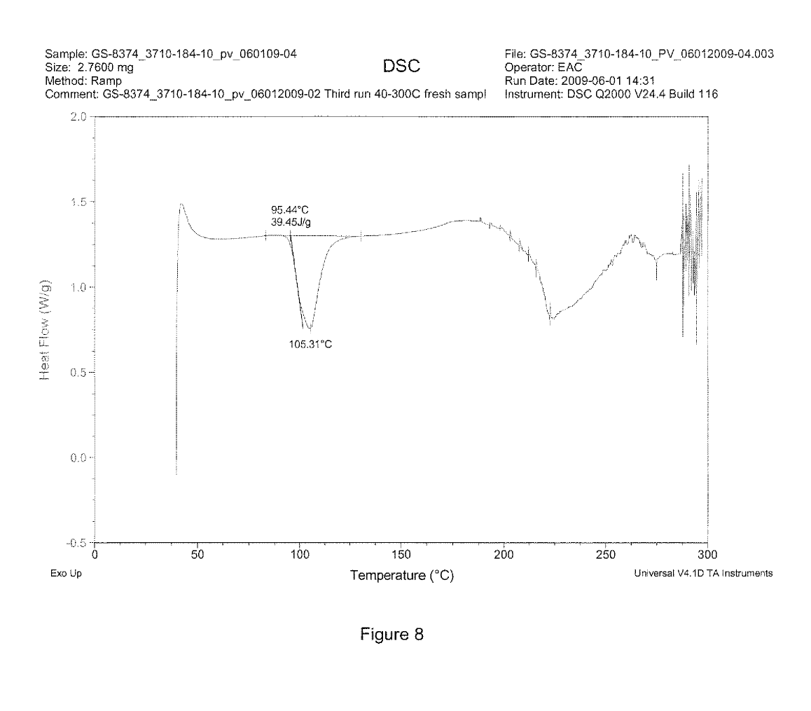

FIG. 9 shows comparison of DSC between Compound Form I and Compound I/L-tartaric acid co-crystal Form A.

FIG. 10 shows thermogravimetric analysis of Compound I/L-tartaric acid co-crystal Form A.

FIG. 11 shows X-ray powder diffraction (XRPD) patterns of Compound I/L-tartaric acid co-crystal Form B.

FIG. 12 shows differential scanning calorimetry (DSC) of Compound a—tartaric acid co-crystal Form B.

FIG. 13 shows thermogravimetric analysis of Compound I/L-tartaric acid co-crystal Form B.

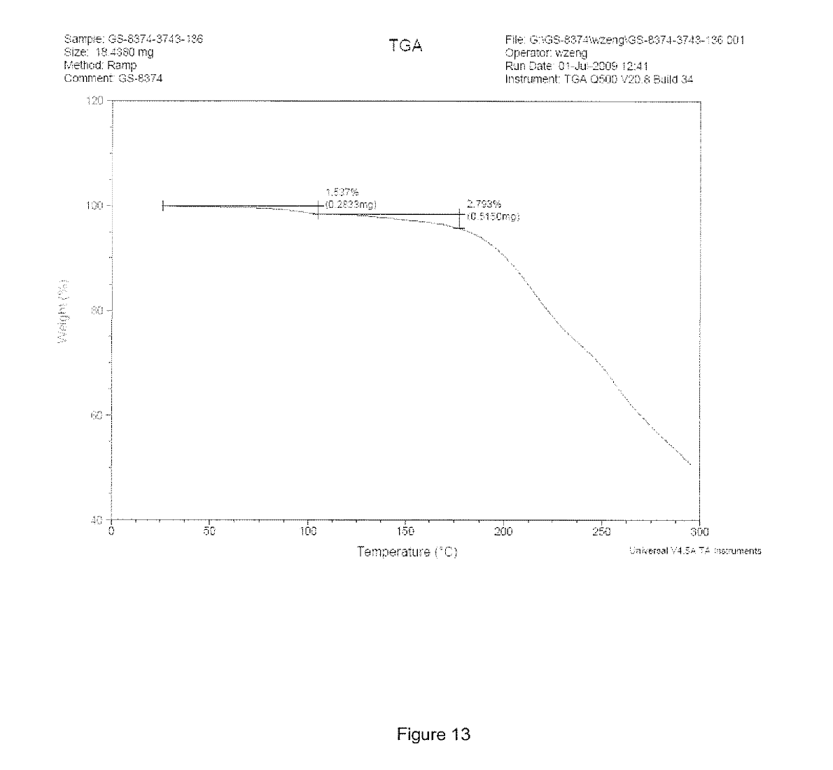

FIG. 14 shows X-ray powder diffraction (XRPD) patterns of Compound I/malonic acid co-crystal form.

FIG. 15 shows differential scanning calorimetry (DSC) of Compound I/malonic acid co-crystal form.

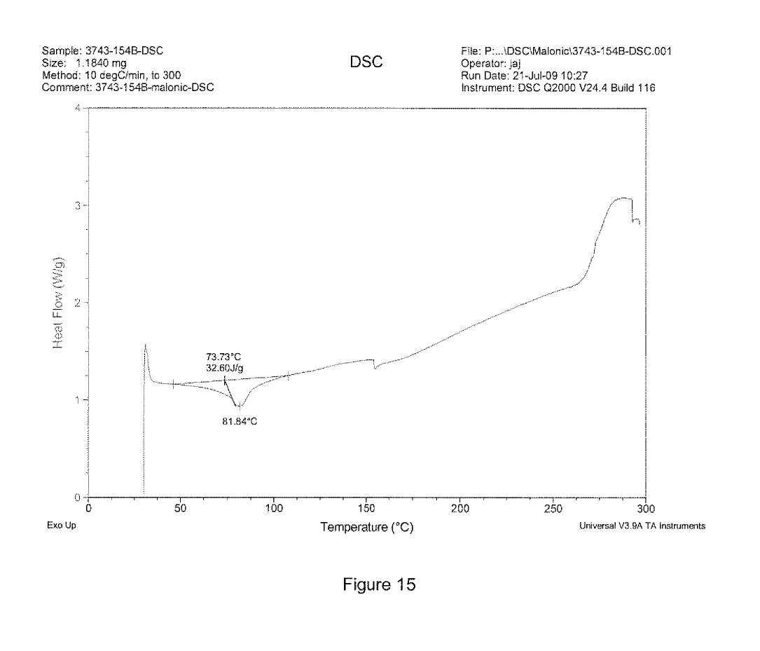

FIG. 16 shows X-ray powder diffraction ((XRPD)) patterns of Compound I/L-malic acid co-crystal form.

FIG. 17 shows differential scanning calorimetry (DSC) of Compound I/L, malic acid co-crystal form.

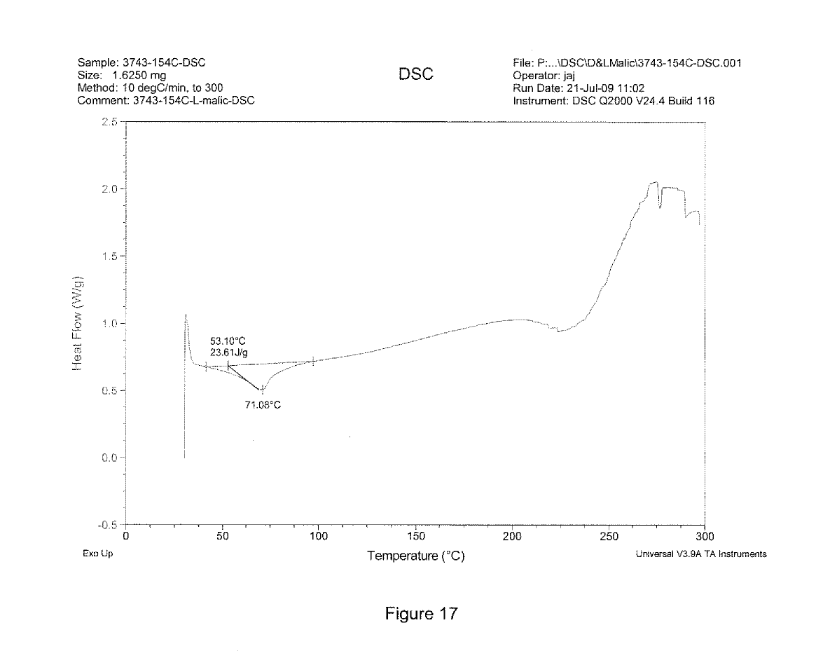

FIG. 18 shows X-ray powder diffraction (XRPD) patterns of Compound I/D-malic acid co-crystal form.

FIG. 19 shows differential scanning calorimetry (DSC) of Compound I/D-malic acid co-crystal form.

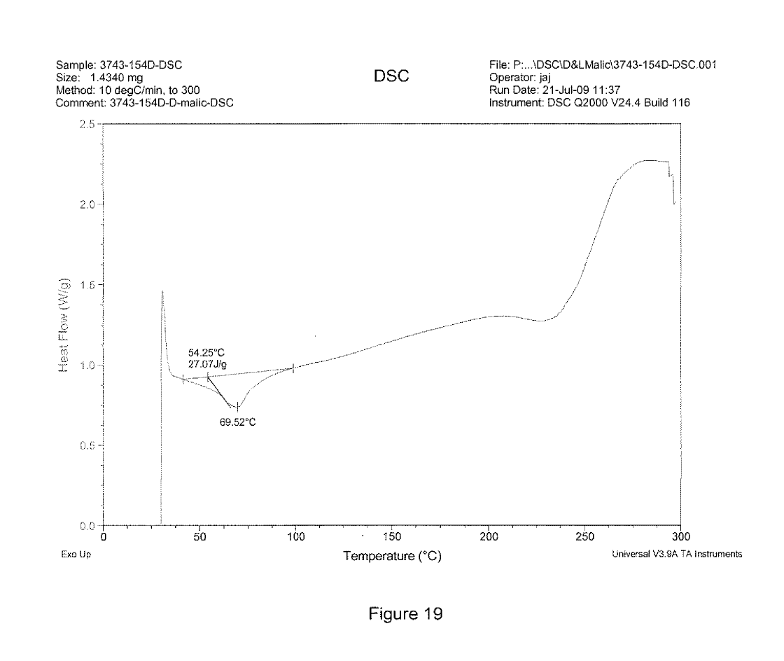

FIG. 20 shows X-ray powder diffraction (XRPD) patterns of Compound I/benzoic acid co-crystal form.

FIG. 21 shows differential scanning calorimetry (DSC) of Compound I/benzoic acid co-crystal form.

FIG. 22 shows X-ray powder diffraction (XRPD) pattern of Compound I/D-tartaric acid co-crystal form

### DETAILED DESCRIPTION OF THE INVENTION

For purposes of interpreting this specification, the following definitions will apply and whenever appropriate, terms used in the singular will also include the plural and vice versa.

As used herein, the term “substantially” refers to degree of variations of +/− by about 1%, about 5%, about 10%, about 15% or about 20%.

As used herein, the term “substantially pure” with respect to a particular polymorphic form of a compound, means that the polymorph form contains about less than 30%, or about less than 20%, or about less than 15%, or about less than 10%, or about less than 5%, or about less than 1% by weight of impurities, such impurities may include other polymorphic forms of the same compound.

As used herein, the term “about” when used in association with a measurement, or used to modify a value, a unit, a constant, or a range of values, refers to variations of +/−3%. A person of ordinary skill in the art would understand that such use of the term “about” does not affect the operation of the invention or its patentability.

As used herein, the term “co-crystal” refers to a crystalline material comprising two or more unique solids at room temperature, at least one of which is a co-crystal former, each containing distinctive physical characteristics, such as structure, melting point and heats of fusion. The co-crystal can be constructed through several modes of non-covalent molecular recognition including hydrogen-bonding, pi-stacking, guest-host complexation and Van-Der-Waals interactions between the molecules of the solids. Co-crystal is different from solvate.

However, the co-crystal may include one or more solvate molecules in the crystalline lattice.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (See, for example, Remington's Pharmaceutical Sciences, 18th Ed. Mack Printing Company, 1990, pp. 1289-1329, incorporated herein by reference.). Except insofar as any conventional carrier is incompatible with the active ingredient, its use in the therapeutic or pharmaceutical compositions is contemplated.

The term “therapeutically effective amount” of the polymorphic compound of the present invention refers to an amount of the polymorphic compound of the present invention that will elicit the biological or medical response of a subject, or ameliorate symptoms, slow or delay disease onset, progression, or prevent a disease, etc. In a particular embodiment, the “therapeutically effective amount” refers to the amount that inhibits or reduces expression or activity of HIV protease, or treats or provides benefits in the treatment or management of HIV infection or conditions associated with HIV infection in a subject.

As used herein, the term “subject” refers to an animal. Preferably, the animal is a mammal. A subject also refers to for example, primate (e.g., human), cow, sheep, goat, horse, dog, cat, rabbit, rat, mouse, fish, bird and the like. In a preferred embodiment, the subject is a human.

The present invention provides novel crystalline forms of (3R,3aS,6aR)-hexahydrofuro[2,3-b]furan-3-yl (1S,2R)-(1-(4-[(diethoxyphosphoryl)methoxy]phenyl}-3-hydroxy-4-[4-methoxy-N-(2-methylpropyl)benzenesulfonamido]butan-2-yl)carbamate (Compound I) represented by the structure below,

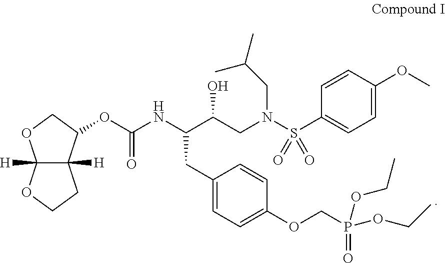

Non-limiting examples include for example, crystalline form I (Form I) and co-crystal forms of Compound I comprising Compound I and various co-formers.

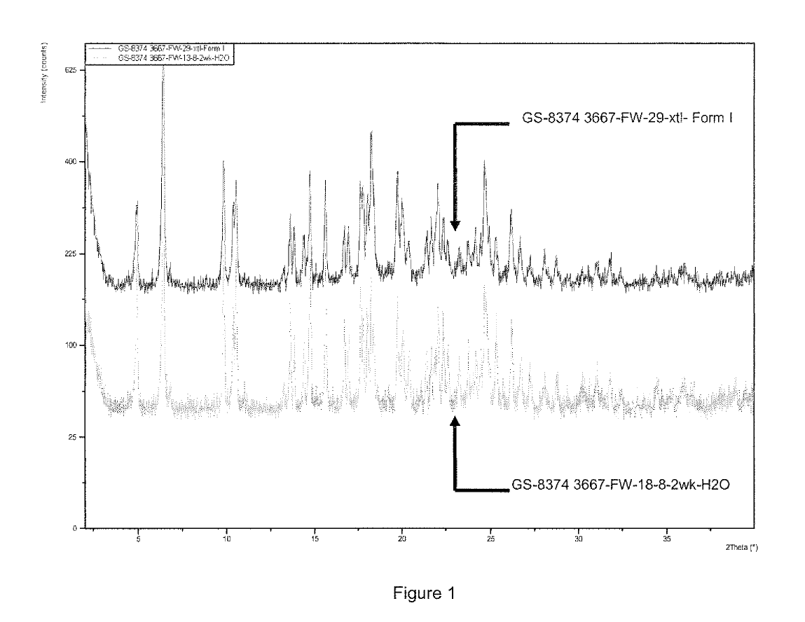

In one aspect, the present invention provides a novel crystalline form of Compound I named Form I. Form I, has a X-ray powder diffraction pattern comprising characteristic peaks at diffraction angles expressed in degrees 2-theta of about 4.9, 6.4, 9.8, 9.8, 10.4, 10.5, 13.6, 14.7, 15.6, 17.6, 18.3, and 24.7. In one embodiment, Form I has substantially the same X-ray powder diffraction pattern shown in FIG. 1. In another embodiment, Form I is a substantially pure crystalline form. In a further embodiment, Form I of the present invention contains about less than 30%, or about less than 20%, or about less than 15%, or about less than 10%, or about less than 5%, or about less than 1% by weight of impurities.

Yet in another embodiment, Form I of the present invention has about 1.4% to 2.4% by weight of water.

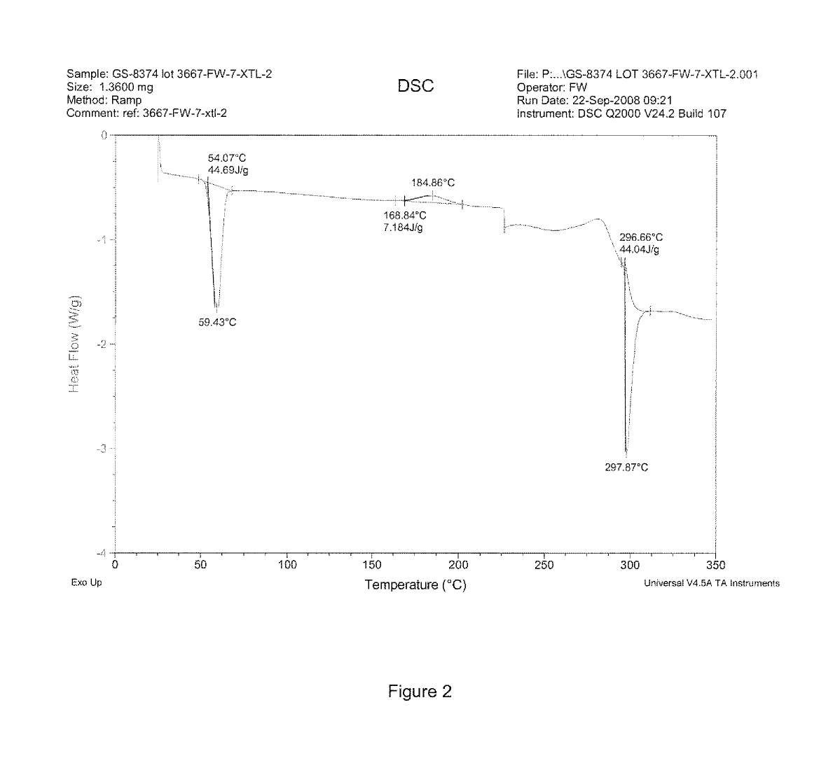

Yet in another embodiment, Form I of the present invention has a differential scanning calorimetry (DSC) peak with an onset of about 54° C., as shown in FIG. 2.

Form I of the present invention offers better properties than the amorphous form of Compound I. For example, Form I is less hygroscopic than the amorphous form. Furthermore, Form I is more pure and stable than the amorphous form, and is easier to handle and process than the amorphous form. Therefore, the present invention is more commercially viable for manufacturing and isolation processes, and better suited for developing pharmaceutical dosage forms.

In another aspect, the present invention provides methods for preparing Form I of the present invention, said methods include dissolving (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate (Compound I) in either an aqueous solvent or an organic solvent, or an aqueous-organic mixed solvent to form a slurry, removing the solvent, and drying the solids. In one embodiment, Compound I can be mixed or dissolved in either aqueous or organic solvents to form a slurry. The resulting slurry is a substantially pure polymorph of Form I. In one embodiment, the aqueous solvent can be water. In another embodiment, the aqueous-organic mixed solvent can be a mixture of water and one or two solvent selected from methanol, ethanol, isopropanol, diisopropyl ether, toluene, or heptane. In a further embodiment, the solvent is methanol/water, ethanol/water, or diisopropyl ether/toluene/water, isopropanol/water, isopropyl acetate/heptane/water, methyl t-butyl ether/heptane/water, toluene/heptane/water.

In one embodiment, Form I of the present invention may involve addition of at least one seed of the crystalline form for accelerating or optimizing formation of the polymorph of the present invention.

In another aspect, the present invention provides co-crystals of Compound I comprising Compound I and a co-crystal former. In one embodiment, the co-crystal former is selected from L-tartaric acid, D-tartaric acid, malonic acid, L-malic acid, D-malic acid or benzoic acid, and the like.

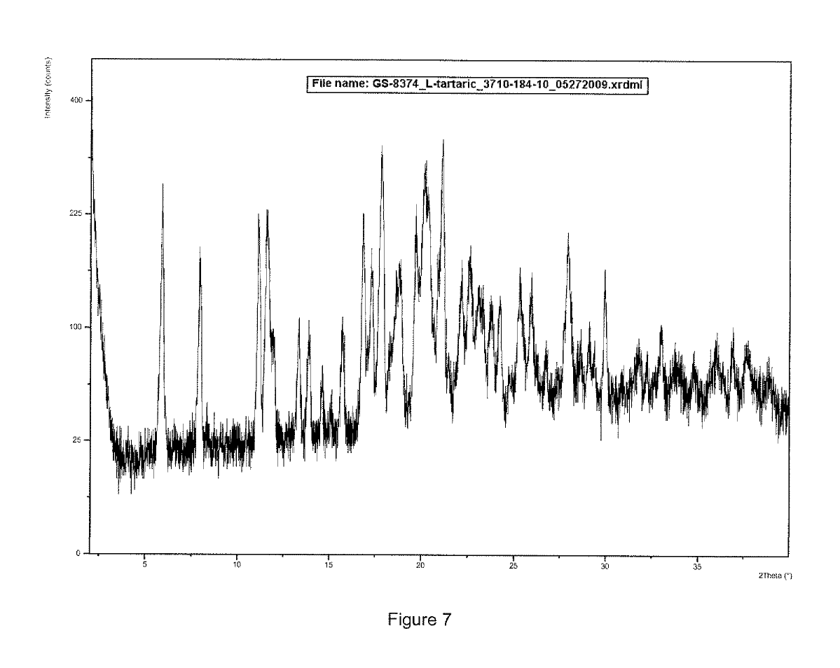

In another embodiment, the co-crystal Form A of the present invention comprises Compound I and L-tartaric acid. Preferably said co-crystal is characterized by X-ray powder diffraction pattern peaks at diffraction angles expressed in degrees 2-theta of about 5.9, 7.9, 11.1, 11.6, 13.3, 13.9, 15.7, 16.8 and 17.9. Also preferably said co-crystal has substantially same X-ray powder diffraction pattern shown in FIG. 7 (Form A).

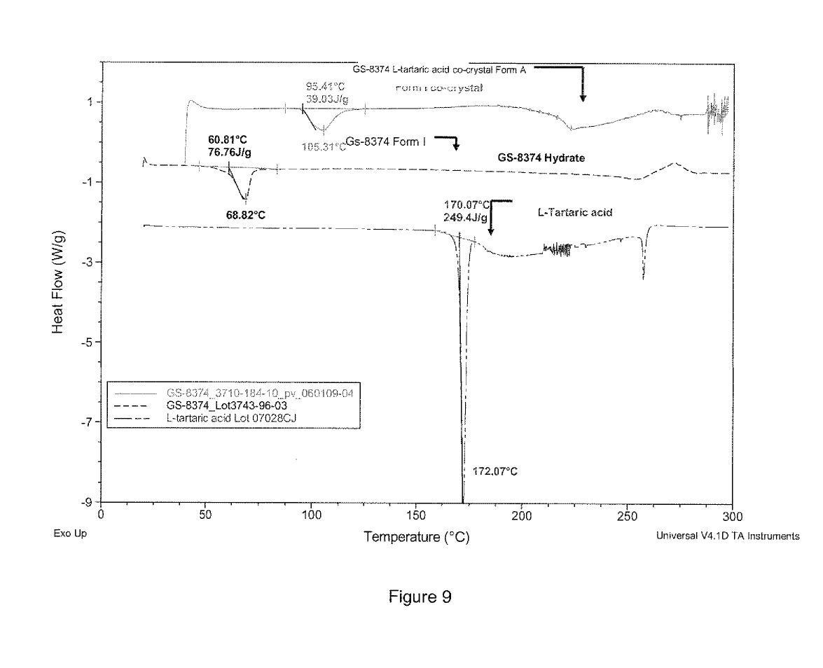

In another embodiment, the co-crystal Form A of the present invention comprises Compound I and L-tartaric acid, said co-crystal having a differential scanning calorimetry (DSC) endothermic peak with an onset of about 95° C., as shown in FIG. 9.

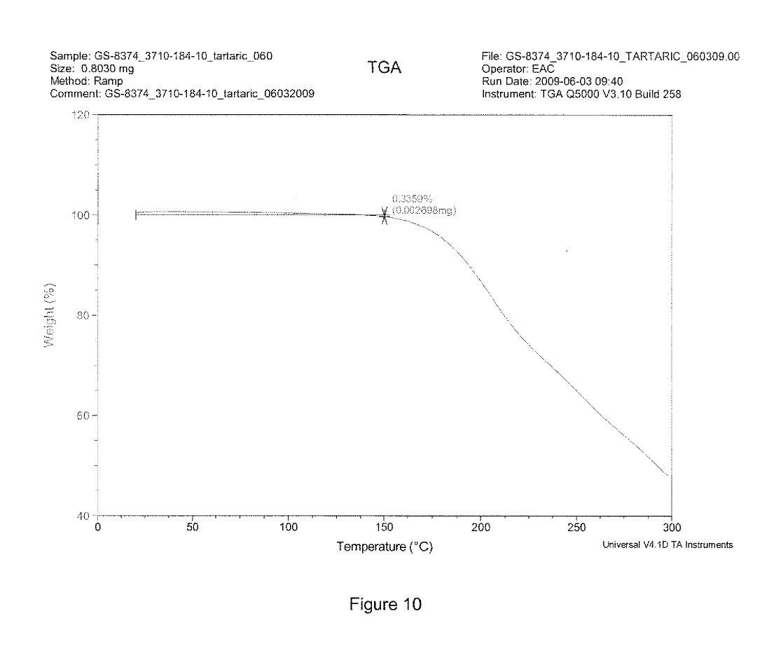

In another embodiment, the co-crystal Form A of the present invention comprises Compound I and L-tartaric acid, said co-crystal having TGA thermogram shown in FIG. 10.

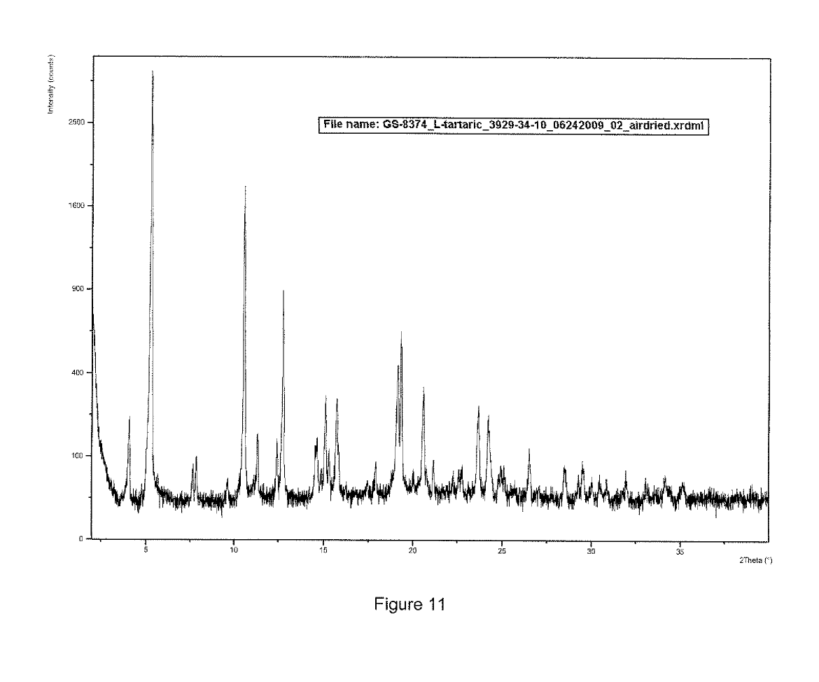

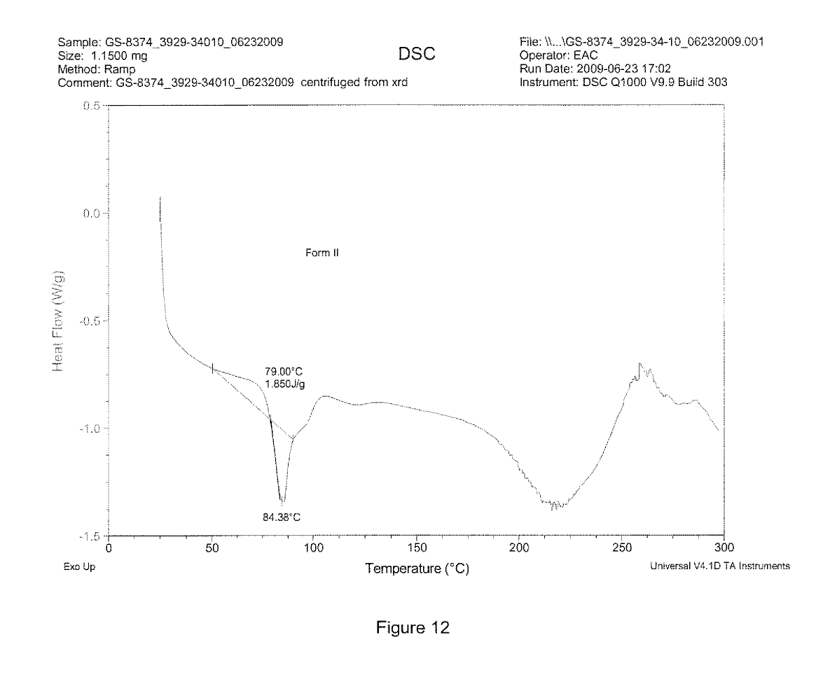

In another embodiment, the present invention provides another co-crystal form of Compound I and L-tartaric acid, Form B, said co-crystal is characterized by X-ray powder diffraction pattern peaks at diffraction angels expressed in degrees 2-theta of about 4.1, 5.3, 7.7, 9.6, 10.6, 11.3, 12.4, 15.1 15.7, 19.1, and 19.3. Preferably said co-crystal has substantially same X-ray powder diffraction pattern shown in FIG. 11 (Form B). Also preferably, said co-crystal has a differential scanning calorimetry (DSC) endothermic peak with an onset of about 79° C. as shown in FIG. 12.

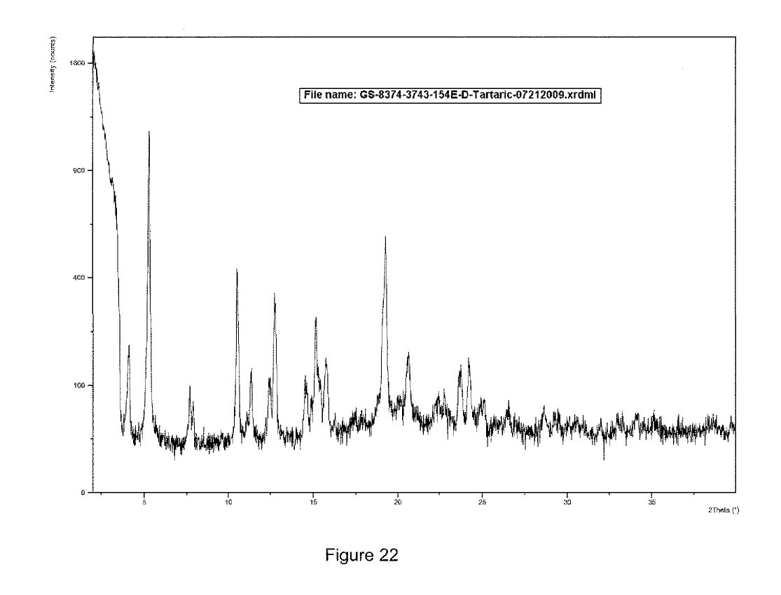

In another embodiment, the co-crystal of the present invention comprises Compound I and D-tartaric acid. Preferably said co-crystal is characterized by X-ray powder diffraction pattern peaks at diffraction angels expressed in degrees 2-theta of about 4.1, 5.3, 7.7, 7.9, 10.6, 11.4, 12.5, 12.8, 14.6 and 15.2. Also preferably said co-crystal has substantially same X-ray powder diffraction pattern shown in FIG. 22.

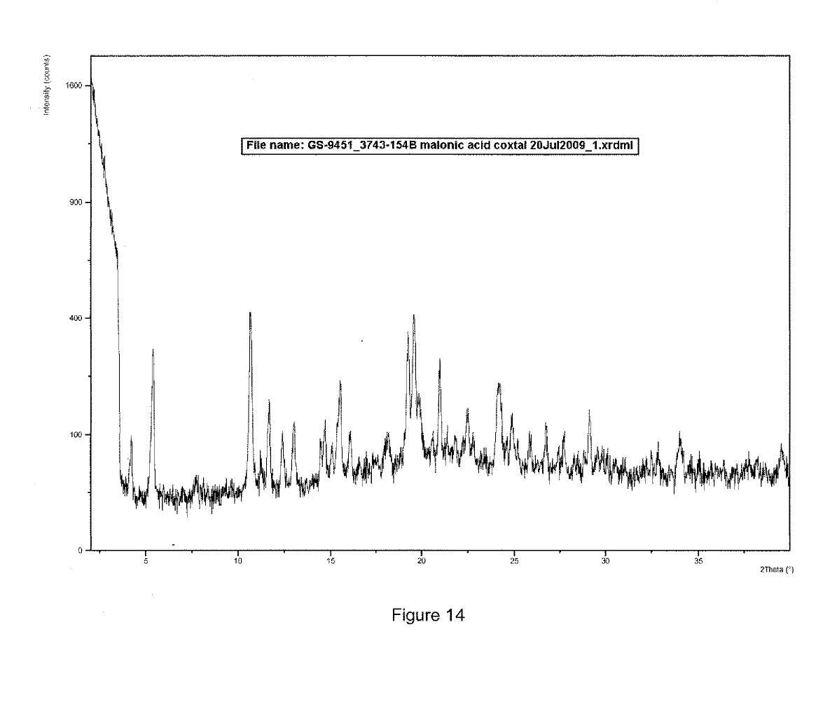

In another embodiment, the co-crystal of the present invention comprises Compound I and malonic acid. Preferably said co-crystal is characterized by X-ray powder diffraction pattern peaks at diffraction angels expressed in degrees 2-theta of about 4.2, 5.4, 10.7, 11.6, 12.4, 13.0, 15.4, 16.1 and 19.5. Also preferably said co-crystal has substantially same X-ray powder diffraction pattern shown in FIG. 14.

In another embodiment, the co-crystal of the present invention comprises Compound I and malonic acid, said co-crystal having a differential scanning calorimetry (DSC) endothermic peak with an onset of about 73° C.

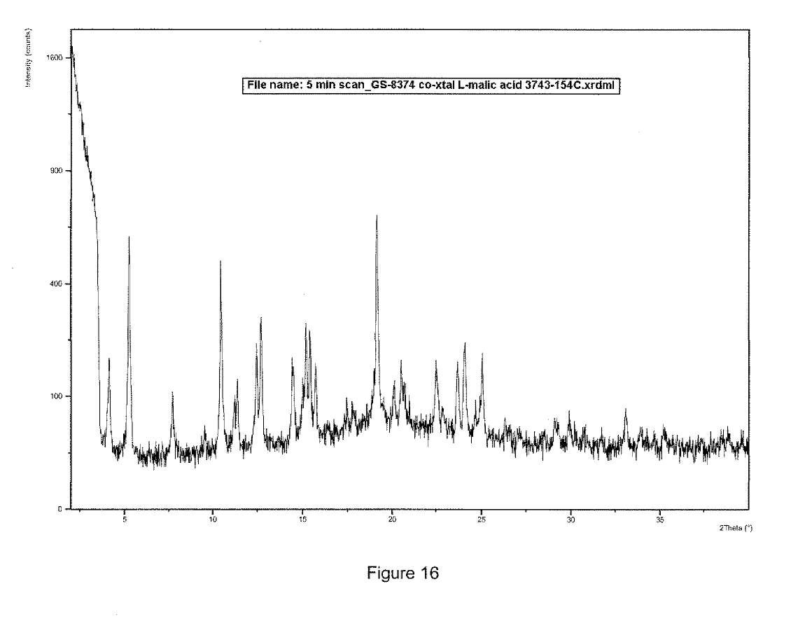

In another embodiment, the co-crystal of the present invention comprises Compound I and L-malic acid. Preferably said co-crystal is characterized by X-ray powder diffraction pattern peaks at diffraction angels expressed in degrees 2-theta of about 4.1, 5.3, 7.7, 10.5, 11.4, 12.7, 14.5, 15.2 and 19.2. Also preferably said co-crystal has substantially same X-ray powder diffraction pattern shown in FIG. 16.

In another embodiment, the co-crystal of the present invention comprises Compound I and L-malic acid, said co-crystal having a differential scanning calorimetry (DSC) endothermic peak with an onset of about 53° C.

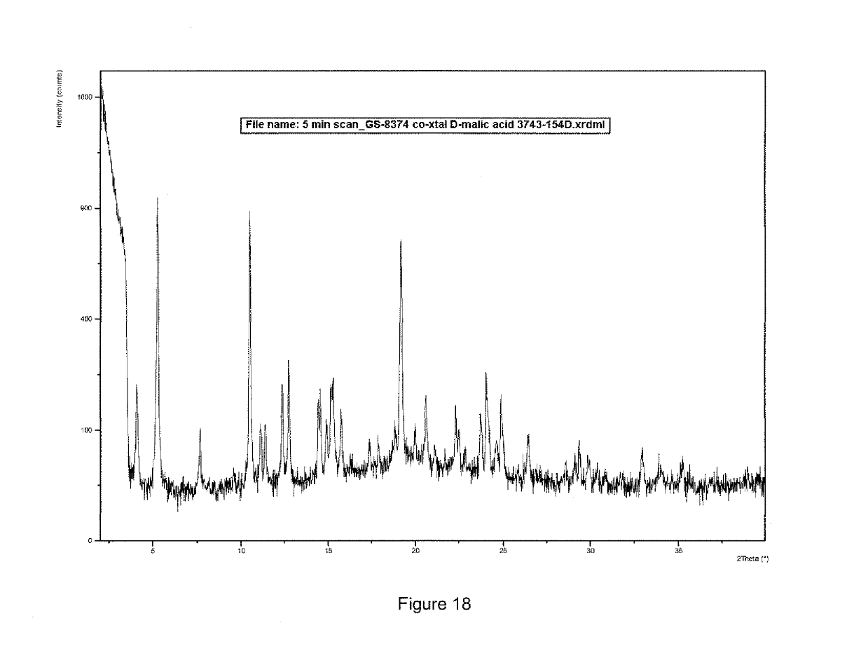

In another embodiment, the co-crystal of the present invention comprises Compound I and D-malic acid. Preferably said co-crystal is characterized by X-ray powder diffraction pattern peaks at diffraction angels expressed in degrees 2-theta of about 4.1, 5.3, 7.6, 10.5, 11.1, 12.4, 12.7, 14.6, 15.5 and 19.2. Also preferably said co-crystal has substantially same X-ray powder diffraction pattern shown in FIG. 18.

In another embodiment, the co-crystal of the present invention comprises Compound I and D-malic acid, said co-crystal having a differential scanning calorimetry (DSC) endothermic peak with an onset of about 54° C.

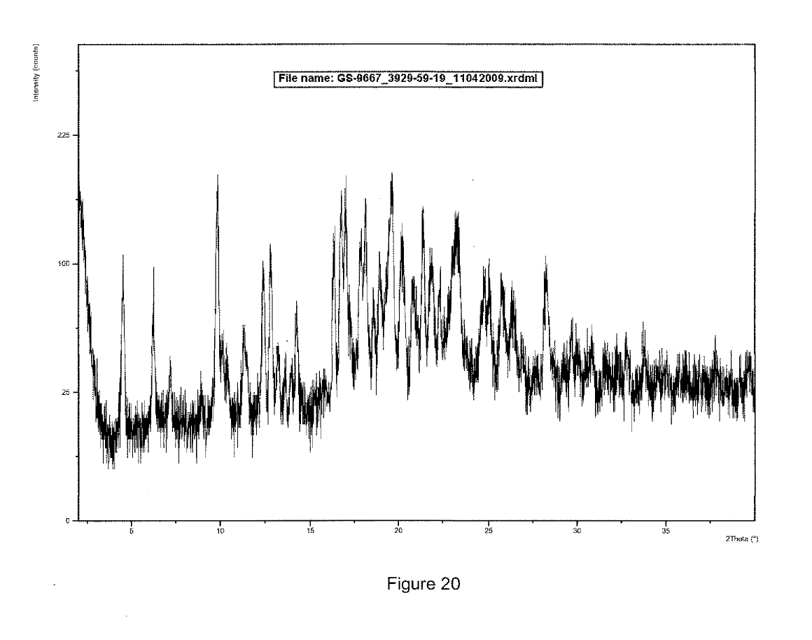

In another embodiment, the co-crystal of the present invention comprises Compound I and benzoic acid. Preferably said co-crystal is characterized by X-ray powder diffraction pattern peaks at diffraction angels expressed in degrees 2-theta of about 4.5, 6.3, 7.2, 9.9, 11.4, 12.4, 12.8, 14.3, 16.4 and 16.8 Also preferably said co-crystal has substantially same X-ray powder diffraction pattern shown in FIG. 20.

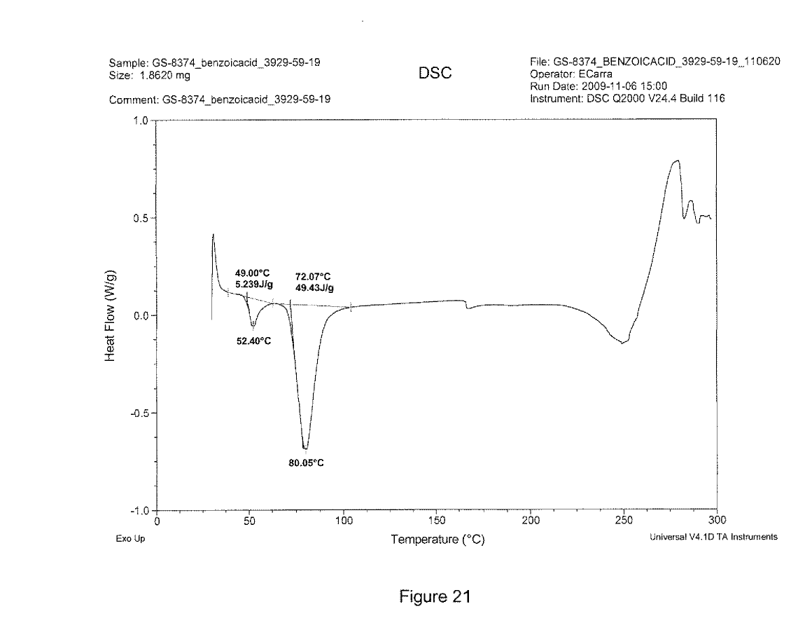

In another embodiment, the co-crystal of the present invention comprises Compound I and benzoic acid, said co-crystal having a differential scanning calorimetry (DSC) endothermic peak with an onset of about 72° C. as shown in FIG. 21.

According to the present invention, the ratio of Compound I to co-crystal formers may be stoichiometric or non-stoichiometric. Non-limiting examples include but not limited to, 1:1, 1:1.5, 1.5:1, 1:2, 2:1 and 3:1, etc. In addition, co-crystals with vacancies within the crystalline lattice are included in the present invention. For example, a co-crystal with less than or about 0.01, 0.1, 1, 2, 3, 4, 5, 6, 7, 8, 9, 10, 11, 12, 13, 14, 15, 16, 17, 18, 19 or 20 percent vacancies within the crystalline lattice are included in the present invention. The vacancies can be due to missing Compound I molecules or missing co-crystal former molecules from the crystalline lattice, or both.

Another aspect of the present invention provides methods of preparing co-crystals of Compound I. The methods include solubilizing/contacting Compound I and co-crystal formers in a solvent and applying crystallization conditions to Compound I and co-crystal formers. In one embodiment, co-crystals of Compound I and formers can be obtained by separately dissolving Compound I and a co-crystal former in a solvent or a mixture of solvents and adding one to the other. The co-crystal may then precipitate or crystallize when the solvent mixture is evaporated slowly. The co-crystal may also be obtained by dissolving Compound I and a co-crystal former in the same solvent or a mixture of solvents. In addition, the co-crystal may also be obtained by seeding a saturated solution of Compound I and the former or seeding with a co-crystal.

In one embodiment, solvents used in the above processes of preparing the co-crystals can be but are not limited to, acetone, methanol, ethanol, isopropyl alcohol, ethyl acetate, isopropyl acetate, nitromethane, dichloromethane, chloroform, toluene, propylene glycol, dimethyl sulfoxide (DMSO), dimethyl formamide (DMF), diethyl ether (ether), ethyl formate, hexane, acetonitrile, benzyl alcohol, water, or another organic solvent including alcohols.

The co-crystals of the present invention can be characterized with conventional methods known in the art. Techniques including X-ray diffraction (XRPD), differential scanning calorimetry (DSC), thermogravimetric analysis (TGA), infrared spectroscopy (IR), single crystal X-ray diffraction and Raman spectroscopy, etc. may be used to characterize the co-crystals of the present invention. XRPD can be used to characterize the physical form of the co-crystals by recording their original patterns and monitoring changes in the patterns with time. DSC can be used to detect thermal transitions occurring in the co-crystals as a function of temperature and to determine the melting point of the co-crystals. TGA can be used to investigate the presence of residual solvents in the samples, and to identify the temperature at which decomposition of the co-crystals occur.

It is a surprising discovery that the co-crystals of the present invention give rise to improved properties over Compound I itself with respect to solubility, dissolution, bioavailability, stability, Cₘₐₓ, Tₘₐₓ, processability, hygroscopicity, longer lasting therapeutic plasma concentration, etc. This discovery increases opportunities for one skilled in the art to identify an improved formulation suitable for FDA approval.

In another aspect, the crystalline forms of the present invention can be formulated as a pharmaceutical composition. In one embodiment, the pharmaceutical composition comprises a therapeutically effective amount of the crystalline form(s) of Compound I and a pharmaceutically acceptable carrier or excipient. The pharmaceutical composition can be administered orally, parenterally, by inhalation spray, topically, rectally, nasally, buccally, vaginally or via an implanted reservoir. Oral pharmaceutical composition and dosage form are a preferred dosage form. Preferably, the oral dosage form is a solid dosage form, such as a tablet, a caplet, a hard gelatin capsule, a starch capsule, a hydroxypropyl methylcellulose (“HPMC”) capsule, or a soft elastic gelatin capsule, parenteral pharmaceutical compositions and dosage forms. Other preferred dosage forms include an intradermal dosage form, an intramuscular dosage form, a subcutaneous dosage form, and an intravenous dosage form.

Examples of excipients that can be used in oral dosage forms of the invention include, but are not limited to, binders, stabilizers, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof. Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In another aspect, the present invention provides methods of using the crystalline forms or the pharmaceutical composition containing the crystalline forms of the present invention, or a mixture thereof for therapeutic applications. In one embodiment, the present invention provides a method of inhibiting activity of a HIV protease in a subject comprising administering to the subject a therapeutically effective amount of any of the crystalline forms of the present invention, or the pharmaceutical composition containing any of the crystalline forms of the present invention. In another embodiment, the present invention provides a method of treating HIV infection or conditions associated with HIV infection comprising administering to the subject a therapeutically effective amount of any of the crystalline forms of the present invention, or the pharmaceutical composition thereof.

Additionally, the present invention provides:

Any of the crystalline forms of the present invention for use as a medicament;

use of any of the crystalline forms of the present invention for the preparation of a medicament for inhibiting activity of a HIV protease;

use of the pharmaceutical composition of the present invention for the preparation of a medicament for inhibiting activity of a HIV protease;

use of any of the crystalline forms of the present invention for the preparation of a medicament for treating HIV infection or conditions associated with HIV infection;

use of the pharmaceutical composition of the present invention for the preparation of a medicament for treating HIV infection or conditions associated with HIV infection;

### EXAMPLES

The examples in the sections below will further illustrate the preparation and characterization of the polymorph form of the present invention. They are not intended to limit the scope of the present invention as described herein and as claimed below.

### Example 1

### Crystalline Form Screen

Approximately 15-50 mg of Compound I are weighed and transferred to each of fifteen vials each equipped with a mini magnetic stir bar. The starting material used for this experiment is an amorphous solid based on the X-ray powder diffraction (XRPD) pattern (See FIG. 1). To each vial is added 1 mL of various solvents. The slurries are allowed to stir for two weeks at room temperature (˜22° C.). XRPD characterization of the excess solids were performed at ˜1 week and ˜2 week time points. A sample of 300 μL of the suspension is removed from each vial, transferred to a centrifuge filter (Costar, 0.45 micron) and centrifuged at 1000 rpm for 2 minutes. Solids in the wet cake form are analyzed using XRPD analysis (PANalytical X'Pert PRO X-ray powder diffractometer). This diffractometer uses Cu-Ka radiation and it operates under reflection mode. Scan range is from 2 to 40 degree 2 theta. After air drying the small sample, (XRPD) analysis is performed again to monitor any change in the diffraction pattern between the wet and dry material. TGA is run on the saturated solution to determine the solubility of the equilibrating solids in the solvent. FIG. 1 shows the (XRPD) pattern of polymorph Form I of the present invention.

This screening study has revealed that Compound I transforms from amorphous solid to crystalline material after being stirred in water for ˜5 days. As shown in FIG. 1, the solid material (Compound I water slurry-5 days-wet cake) that is in equilibrium with water has a distinct (XRPD) pattern as compared to the amorphous halo for the starting material. The significant signals in the X-Ray diffraction diagram are shown in Table 1. HPLC analysis of the solid shows that this amorphous to crystalline transformation improved the % AN purity from 98.6% to 99.1%.

TABLE 1
XRPD diffraction peaks data for Compound I Form I

| Peak Position | Peak Height | FWHM | d-spacing |
|---|---|---|---|
| [°2Th.] | [cts] | [°2Th.] | [Å] |
| 4.9 | 173 | 0.05 | 17.9 |
| 6.4 | 387 | 0.07 | 13.8 |
| 9.8 | 143 | 0.08 | 9.0 |
| 9.8 | 72 | 0.08 | 9.0 |
| 10.4 | 62 | 0.05 | 8.5 |
| 10.5 | 140 | 0.08 | 8.4 |
| 13.6 | 59 | 0.06 | 6.5 |
| 13.8 | 36 | 0.08 | 6.4 |
| 14.4 | 23 | 0.09 | 6.2 |
| 14.7 | 170 | 0.05 | 6.0 |
| 15.6 | 119 | 0.06 | 5.7 |
| 16.7 | 43 | 0.09 | 5.3 |
| 17.0 | 31 | 0.06 | 5.2 |
| 17.6 | 72 | 0.06 | 5.0 |
| 18.0 | 40 | 0.05 | 4.9 |
| 18.3 | 75 | 0.06 | 4.9 |
| 19.7 | 44 | 0.05 | 4.5 |
| 20.4 | 18 | 0.13 | 4.4 |
| 21.4 | 16 | 0.13 | 4.2 |
| 22.0 | 35 | 0.08 | 4.0 |
| 22.3 | 40 | 0.08 | 4.0 |
| 23.2 | 11 | 0.14 | 3.8 |
| 23.7 | 12 | 0.19 | 3.7 |
| 24.7 | 53 | 0.19 | 3.6 |
| 25.3 | 32 | 0.13 | 3.5 |
| 26.2 | 32 | 0.09 | 3.4 |
| 26.7 | 14 | 0.19 | 3.3 |
| 27.3 | 11 | 0.19 | 3.3 |
| 28.8 | 9 | 0.25 | 3.1 |

This crystalline form discovery is based on the solvent mediated phase transformation to determine if there is a more stable crystalline form than the amorphous solid. See David J. W. Grant, Victor Young Junior, Chong-Hui Gu, Polymorph Screening: Influence of Solvents on the Rate of Solvent-Mediated Polymorphic Transformation. Journal of Pharmaceutical Sciences, 2001. 90(11): p. 1878-1890; and Jonathan M. Miller, Benjamin M. Coltman, Landon R. Greene, David J. W. Grant, Anthony C. Blackburn, Identifying the Stable Polymorph Early in the Drug Discovery-Development Process. Pharmaceutical Development and Technology, 2005(10): p. 291-297. The interaction between water molecules and Compound I molecules, especially hydrogen bonding between the two types of molecules, appears to support the crystalline structure of the hydrate form. The first crystalline form of Compound I isolated from water is named as Form I.

### Example 2

### Characterization of Form I of Compound I

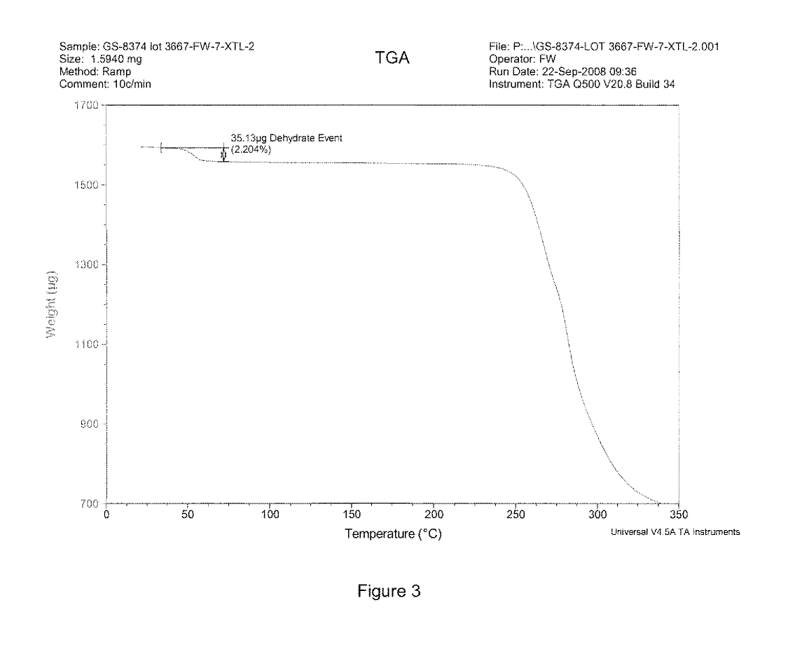

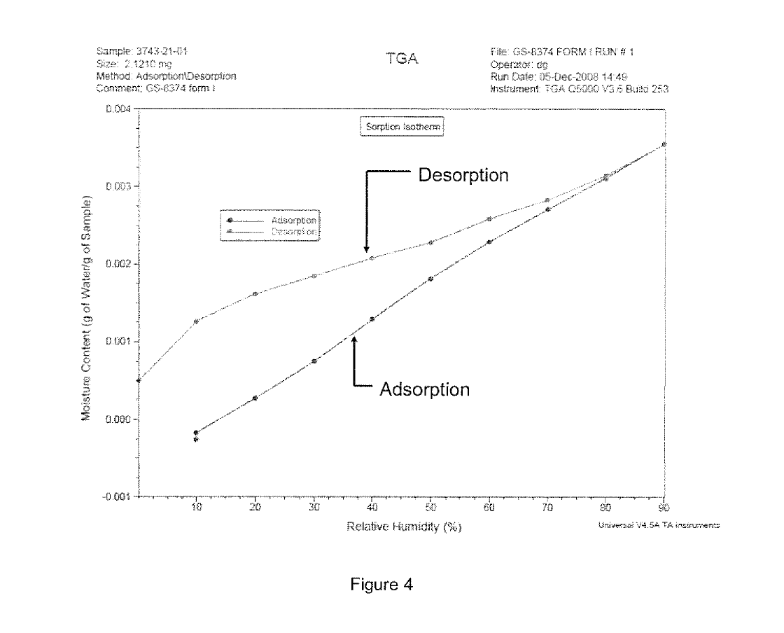

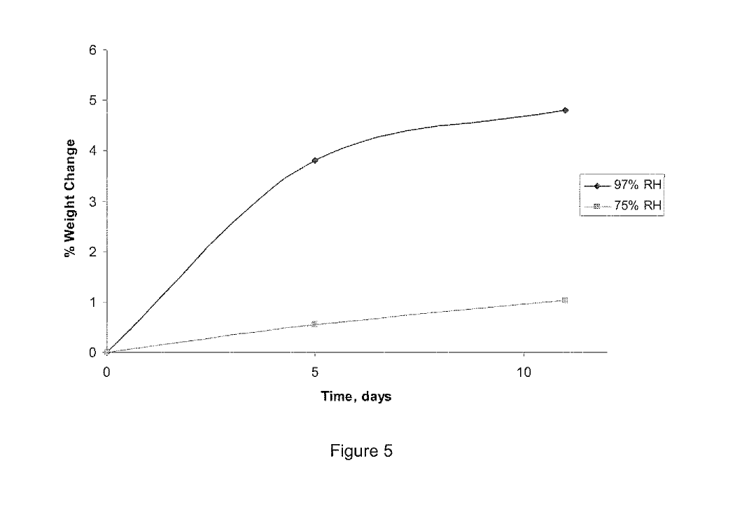

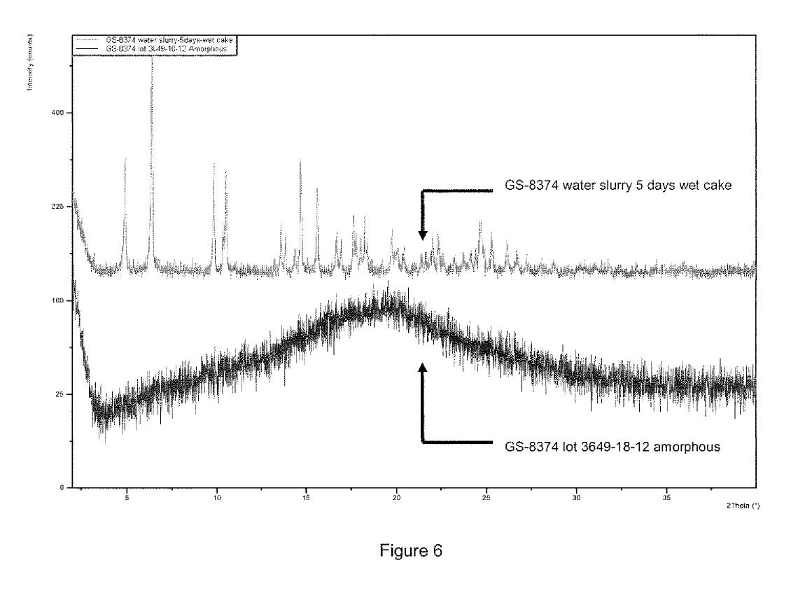

The DSC of Form I (FIG. 2) shows that this crystalline material has a melting point of 54° C. The TGA of Form I (FIG. 3) shows a 2.2% weight loss at ˜50° C., which indicates that this crystalline Form I is a monohydrate as the theoretical water content for a monohydrate is 2.4%. A dynamic vapor sorption experiment indicates that Form I is not hygroscopic; even at 90% relative humidity, only 0.4% water is absorbed(FIG. 4). In comparison, the amorphous material absorbs 4.8% moisture upon exposure to 97% relative humidity, as shown in Table 2 and FIG. 5. Further analysis of additional lots crystallized under similar conditions has demonstrated the consistent formation of a monohydrate of Compound I with the same (XRPD) pattern. Upon extended exposure to water, Form I remains as the same form, as illustrated in FIG. 6. Crystalline Compound I Form I has demonstrated a significant improvement in stability over the amorphous Compound I material, especially considering the crystallinity and low hygroscopicity. Other benefits that crystalline Form I offers include but are not limited to purification and isolation of the crystalline form with respect to the solution and facilitation of storage and handling during the formulation development and manufacturing of drug product.

TABLE 2
Moisture uptake of Compound I amorphous material

| Condition at Room | Weight gain after | Weight gain after |
|---|---|---|
| Temperature | 5 days | 11 days |
| 97% Relative | 3.8% | 4.8% |
| Humidity | | |
| 75% Relative | 0.6% | 1.0% |
| Humidity | | |

### Example 3

Compound I Form I can be crystallized from various solvent and mixed solvent systems. A number of solvent combinations were examined and noted for their ability to produce crystalline Compound I, whereby amorphous Compound I was transferred to each vial containing a magnetic stir bar, and to these vials were added solvent/solvent mixtures These suspensions were then allowed to stir for at least 12 hours. Solvent/solvent mixtures leading to crystalline Form I include but are not limited to: methanol/water, ethanol/water, or diisopropyl ether/toluene/water, isopropanol/water, isopropyl acetate/heptane/water, methyl t-butyl ether/heptane/water, toluene/heptane/water, acetone/water. Two specific processes that produce the crystalline Compound I Form I are provided in Example 4 and Example 5.

### Example 4

EtOH/H₂O system: Compound I amorphous solid is dissolved in ethanol. This solution is added to a stirred suspension of Compound I seed in a mixture of water and ethanol. The mixture is stirred for at least 16 hours. Then the slurry is filtered and dried in a vacuum oven at about 21° C. to afford crystalline Compound I Form I.

### Example 5

Diisopropyl ether/toluene/H₂O system: Amorphous Compound I is dissolved in toluene. This solution is added to a stirred suspension of Compound I crystalline Form I seeds in a mixture of diisopropyl ether, water, and toluene. Following completion of addition, the slurry is stirred at ambient temperature for 1 h. The slurry is then filtered, washed with 2×50 mL portions diisopropyl ether, and dried in vacuum oven at ambient temperature overnight to afford crystalline Compound I Form L.

### Example 6

### Compound I/L-Tartaric Acid Co-Crystal Form A

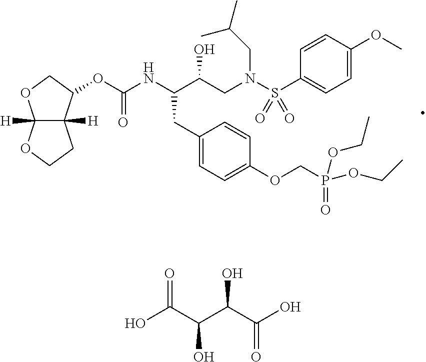

A 20 mL glass reactor is charged with 873 mg Compound I, Form I, 183 mg L-tartaric acid, and 5.0 mL acetonitrile. Compound I dissolved on contact while L-tartaric acid is only partially dissolved.

The reaction vessel is placed in a temperature control unit. The temperature is varied from 21 to 60° C. over 20 minutes; then held at 60° C. for 30 minutes. The temperature is then reduced from 60° C. to 20° C. over 10 hours and finally held at 20° C. for about 17 hours with constant stirring.

At end of temperature cycle, the mixture has become a slightly hazy, light green solution containing a few clear colorless crystals, specifically prisms (closely matching the morphology of L-tartaric acid). The solution is transferred to a 20 mL threaded glass vial and allowed to evaporate unassisted at 21° C. After four days the solution is a mostly clear light green resin with a few crystals imbedded within (prisms) and a few crystals on the walls above the resin (prisms). The vial is capped and held at 21° C.

After 1 more day, a slight opacity is evident in the resin when observed at 14× magnification. The opaque regions originated at the top and spread down into the resin.

After 2 more days, the opacity is heavily spread into the resin, easily visible to naked eye, and likely crystalline products were visible with a 14× monocular. Earlier observed prism forms began to disappear.

After 5 more days, conversion of the resin to a crystalline mass appears nearly complete. No prisms remain. The material, a sticky, resistant mass, is mixed with a metal spatula and allowed to air dry in a fume hood at 21° C.

After 1 day of air drying, the crystalline product is a hard, dry mass. XRPD analysis of the product reveals a highly crystalline material. The XRPD pattern of the product shows similarity to Compound I starting material, but almost no similarity with L-tartaric acid.

The material is dried in a vacuum oven at reduced pressure at 21° C. for three days. XRPD of the solids taken after this drying step shows no change. The product is designated Compound ft-tartaric acid co-crystal Form A. Table 3 shows (XRPD) peaks data of Compound ft-tartaric acid co-crystal Form A.

TABLE 3
The XRPD Peak Data of Compound I/L-
tartaric acid Co-crystal Form A

| | | FWHM | | |
|---|---|---|---|---|
| Pos.[°2Th.] | Height[cts] | [°2Th.] | d-spacing[Å] | Rel. Int.[%] |
| 5.82 | 296.85 | 0.14 | 15.16 | 100.00 |
| 5.84 | 148.42 | 0.14 | 15.16 | 50.00 |
| 7.86 | 185.19 | 0.13 | 11.24 | 62.38 |
| 7.88 | 92.59 | 0.13 | 11.24 | 31.19 |
| 11.06 | 224.80 | 0.12 | 7.99 | 75.73 |
| 11.09 | 112.40 | 0.12 | 7.99 | 37.86 |
| 11.47 | 154.75 | 0.13 | 7.71 | 52.13 |
| 11.50 | 77.38 | 0.13 | 7.71 | 26.07 |
| 11.57 | 176.78 | 0.11 | 7.64 | 59.55 |
| 11.60 | 88.39 | 0.11 | 7.64 | 29.78 |
| 11.82 | 76.96 | 0.22 | 7.48 | 25.93 |
| 11.85 | 38.48 | 0.22 | 7.48 | 12.96 |
| 13.25 | 85.42 | 0.12 | 6.67 | 28.78 |
| 13.29 | 42.71 | 0.12 | 6.67 | 14.39 |
| 13.81 | 75.75 | 0.11 | 6.41 | 25.52 |
| 13.85 | 37.87 | 0.11 | 6.41 | 12.76 |
| 14.53 | 38.33 | 0.09 | 6.09 | 12.91 |
| 14.57 | 19.16 | 0.09 | 6.09 | 6.46 |
| 15.64 | 74.18 | 0.13 | 5.66 | 24.99 |
| 15.68 | 37.09 | 0.13 | 5.66 | 12.49 |
| 16.77 | 174.34 | 0.11 | 5.28 | 58.73 |
| 16.81 | 87.17 | 0.11 | 5.28 | 29.37 |
| 17.07 | 20.99 | 4.00 | 5.19 | 7.07 |
| 17.11 | 10.50 | 4.00 | 5.19 | 3.54 |
| 17.22 | 74.63 | 0.14 | 5.15 | 25.14 |
| 17.26 | 37.31 | 0.14 | 5.15 | 12.57 |
| 17.78 | 178.09 | 0.19 | 4.98 | 60.00 |
| 17.83 | 89.05 | 0.19 | 4.98 | 30.00 |
| 19.62 | 135.33 | 0.12 | 4.52 | 45.59 |
| 19.67 | 67.66 | 0.12 | 4.52 | 22.79 |
| 20.14 | 132.19 | 0.50 | 4.41 | 44.53 |
| 20.19 | 66.10 | 0.50 | 4.41 | 22.27 |
| 20.82 | 101.72 | 0.08 | 4.26 | 34.27 |
| 20.87 | 50.86 | 0.08 | 4.26 | 17.13 |
| 21.04 | 234.10 | 0.12 | 4.22 | 78.86 |
| 21.09 | 117.05 | 0.12 | 4.22 | 39.43 |
| 22.04 | 64.83 | 0.16 | 4.03 | 21.84 |
| 22.09 | 32.42 | 0.16 | 4.03 | 10.92 |
| 22.51 | 90.35 | 0.26 | 3.95 | 30.44 |
| 22.57 | 45.17 | 0.26 | 3.95 | 15.22 |
| 23.18 | 41.62 | 0.43 | 3.83 | 14.02 |
| 23.24 | 20.81 | 0.43 | 3.83 | 7.01 |
| 23.91 | 12.57 | 4.00 | 3.72 | 4.24 |
| 23.97 | 6.29 | 4.00 | 3.72 | 2.12 |
| 24.11 | 58.72 | 0.12 | 3.69 | 19.78 |
| 24.17 | 29.36 | 0.12 | 3.69 | 9.89 |
| 25.26 | 52.54 | 0.36 | 3.52 | 17.70 |
| 25.32 | 26.27 | 0.36 | 3.52 | 8.85 |
| 25.84 | 59.15 | 0.10 | 3.44 | 19.92 |
| 25.91 | 29.57 | 0.10 | 3.44 | 9.96 |
| 26.02 | 14.44 | 4.00 | 3.42 | 4.87 |
| 26.09 | 7.22 | 4.00 | 3.42 | 2.43 |
| 27.87 | 81.31 | 0.25 | 3.20 | 27.39 |
| 27.94 | 40.66 | 0.25 | 3.20 | 13.70 |
| 29.38 | 14.55 | 1.16 | 3.04 | 4.90 |
| 29.45 | 7.27 | 1.16 | 3.04 | 2.45 |
| 29.85 | 60.71 | 0.11 | 2.99 | 20.45 |
| 29.92 | 30.36 | 0.11 | 2.99 | 10.23 |
| 32.17 | 9.05 | 3.50 | 2.78 | 3.05 |
| 32.26 | 4.52 | 3.50 | 2.78 | 1.52 |
| 32.67 | 13.21 | 0.63 | 2.74 | 4.45 |
| 32.76 | 6.60 | 0.63 | 2.74 | 2.23 |
| 32.92 | 41.64 | 0.08 | 2.72 | 14.03 |
| 33.01 | 20.82 | 0.08 | 2.72 | 7.01 |
| 34.01 | 10.02 | 2.86 | 2.63 | 3.37 |
| 34.10 | 5.01 | 2.86 | 2.63 | 1.69 |
| 35.53 | 20.95 | 0.23 | 2.52 | 7.06 |
| 35.62 | 10.47 | 0.23 | 2.52 | 3.53 |
| 35.93 | 44.90 | 0.06 | 2.50 | 15.13 |
| 36.02 | 22.45 | 0.06 | 2.50 | 7.56 |
| 36.61 | 12.60 | 1.35 | 2.45 | 4.25 |
| 36.70 | 6.30 | 1.35 | 2.45 | 2.12 |
| 37.61 | 25.50 | 0.20 | 2.39 | 8.59 |
| 37.71 | 12.75 | 0.20 | 2.39 | 4.30 |
| 38.87 | 12.94 | 0.45 | 2.32 | 4.36 |
| 38.97 | 6.47 | 0.45 | 2.32 | 2.18 |

Form A co-crystal is also generated from two other solvent systems: methanol, and acetonitrile/ethanol 9:1 (v/v). In both of these examples, the mixtures are concentrated to the resin state prior to crystallization.

### Example 7

### Compound I/L-Tartaric Acid Co-Crystal Form B

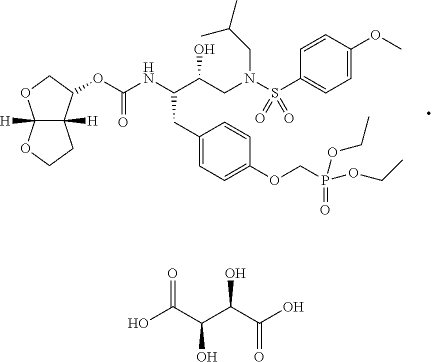

A 20 mL glass reactor equipped with stir bar is charged with 742.2 mg Compound I and 153.3 mg L-tartaric acid (co-former) and 1.0 mL 2-propanol/ethanol (1:1, v/v). The vial is stirred at 21° C. Compound I dissolves rapidly; the co-former dissolves slowly (Compound I and co-former are distinguishable based on their distinctive morphology and density). The mixture is stirred at 70° C. for ˜5 minutes (to completely dissolve the co-former) yielding a homogeneous clear colorless solution. The solution is slowly cooled to 21° C. with stirring.

After 3 days with slow stirring, the mixture is a viscous, clear, colorless solution. To this solution is added ˜1-2 mg authentic Compound I/L-tartaric acid co-crystal seeds Form A. The seeds persists, and after approximately 5 minutes of stirring at 21° C., a noticeable increase in cloudiness is observed in the vessel. This cloudiness notably increases following an additional 10 minutes stirring.

The mixture is allowed to stir at 21° C. for approximately 90 minutes, at which time, the turbidity no longer appears to increase. At this time, to the mixture is added a second dose of approximately 1-2 mg of seeds. Stirring is continued at 21° C. while capped.

After approximately 18 hours, a heavy, white suspension is stirring in the vial. The slurry is first sampled for XRPD as a neat sample and later also after centrifugation. Stirring at 21° C. is maintained. XRPD spectra are obtained for the isolated solids. Solid is isolated as Compound I/L-tartaric acid co-crystal Form B by centrifugation/filtration. Table 4 shows (XRPD) peaks data of Compound III-tartaric acid co-crystal Form B.

TABLE 4
The XRPD Peak Data of Compound I/L-
tartaric acid Co-crystal Form 8

| | | FWHM | | |
|---|---|---|---|---|
| Pos.[°2Th.] | Height[cts] | [°2Th.] | d-spacing[Å] | Rel. Int.[%] |
| 4.08 | 240.92 | 0.08 | 21.65 | 7.89 |
| 4.09 | 120.46 | 0.08 | 21.65 | 3.95 |
| 5.30 | 3051.62 | 0.08 | 16.65 | 100.00 |
| 5.32 | 1525.81 | 0.08 | 16.65 | 50.00 |
| 7.68 | 48.08 | 0.07 | 11.51 | 1.58 |
| 7.70 | 24.04 | 0.07 | 11.51 | 0.79 |
| 7.87 | 62.32 | 0.07 | 11.22 | 2.04 |
| 7.89 | 31.16 | 0.07 | 11.22 | 1.02 |
| 9.64 | 25.29 | 0.11 | 9.17 | 0.83 |
| 9.66 | 12.65 | 0.11 | 9.17 | 0.41 |
| 10.57 | 1695.43 | 0.07 | 8.37 | 55.56 |
| 10.59 | 847.71 | 0.07 | 8.37 | 27.78 |
| 11.34 | 129.11 | 0.07 | 7.79 | 4.23 |
| 11.37 | 64.55 | 0.07 | 7.79 | 2.12 |
| 12.43 | 101.89 | 0.07 | 7.12 | 3.34 |
| 12.46 | 50.94 | 0.07 | 7.12 | 1.67 |
| 12.77 | 792.35 | 0.07 | 6.93 | 25.96 |
| 12.80 | 396.17 | 0.07 | 6.93 | 12.98 |
| 14.56 | 89.24 | 0.07 | 6.08 | 2.92 |
| 14.60 | 44.62 | 0.07 | 6.08 | 1.46 |
| 14.67 | 112.84 | 0.06 | 6.04 | 3.70 |
| 14.70 | 56.42 | 0.06 | 6.04 | 1.85 |
| 15.12 | 224.20 | 0.08 | 5.86 | 7.35 |
| 15.15 | 112.10 | 0.08 | 5.86 | 3.67 |
| 15.32 | 67.51 | 0.04 | 5.78 | 2.21 |
| 15.36 | 33.75 | 0.04 | 5.78 | 1.11 |
| 15.68 | 50.75 | 1.44 | 5.65 | 1.66 |
| 15.72 | 25.38 | 1.44 | 5.65 | 0.83 |
| 15.74 | 204.72 | 0.07 | 5.63 | 6.71 |
| 15.78 | 102.36 | 0.07 | 5.63 | 3.35 |
| 17.90 | 49.21 | 0.07 | 4.95 | 1.61 |
| 17.95 | 24.61 | 0.07 | 4.95 | 0.81 |
| 19.11 | 354.34 | 0.12 | 4.64 | 11.61 |
| 19.16 | 177.17 | 0.12 | 4.64 | 5.81 |
| 19.31 | 577.81 | 0.06 | 4.59 | 18.93 |
| 19.36 | 288.91 | 0.06 | 4.59 | 9.47 |
| 20.24 | 6.28 | 2.57 | 4.38 | 0.21 |
| 20.29 | 3.14 | 2.57 | 4.38 | 0.10 |
| 20.58 | 276.33 | 0.08 | 4.31 | 9.06 |
| 20.63 | 138.17 | 0.08 | 4.31 | 4.53 |
| 21.16 | 60.44 | 0.05 | 4.20 | 1.98 |
| 21.21 | 30.22 | 0.05 | 4.20 | 0.99 |
| 22.29 | 3.71 | 0.00 | 3.99 | 0.12 |
| 22.35 | 1.85 | 0.00 | 3.99 | 0.06 |
| 22.70 | 30.39 | 0.00 | 3.91 | 1.00 |
| 22.76 | 15.19 | 0.00 | 3.91 | 0.50 |
| 23.66 | 201.51 | 0.10 | 3.76 | 6.60 |
| 23.72 | 100.75 | 0.10 | 3.76 | 3.30 |
| 24.23 | 157.18 | 0.12 | 3.67 | 5.15 |
| 24.30 | 78.59 | 0.12 | 3.67 | 2.58 |
| 25.04 | 19.82 | 0.57 | 3.55 | 0.65 |
| 25.10 | 9.91 | 0.57 | 3.55 | 0.32 |
| 26.53 | 87.96 | 0.06 | 3.36 | 2.88 |
| 26.60 | 43.98 | 0.06 | 3.36 | 1.44 |
| 28.51 | 53.13 | 0.11 | 3.13 | 1.74 |
| 28.58 | 26.57 | 0.11 | 3.13 | 0.87 |
| 29.46 | 42.95 | 0.09 | 3.03 | 1.41 |
| 29.54 | 21.48 | 0.09 | 3.03 | 0.70 |
| 31.05 | 2.85 | 2.14 | 2.88 | 0.09 |
| 31.13 | 1.42 | 2.14 | 2.88 | 0.05 |
| 31.95 | 25.82 | 0.05 | 2.80 | 0.85 |
| 32.03 | 12.91 | 0.05 | 2.80 | 0.42 |
| 33.08 | −33.58 | 0.02 | 2.71 | −1.10 |
| 33.16 | −16.79 | 0.02 | 2.71 | −0.55 |
| 34.17 | 23.51 | 0.24 | 2.62 | 0.77 |
| 34.25 | 11.76 | 0.24 | 2.62 | 0.39 |
| 35.15 | 19.25 | 0.16 | 2.55 | 0.63 |
| 35.24 | 9.63 | 0.16 | 2.55 | 0.32 |
| 38.50 | 2.35 | 4.00 | 2.34 | 0.08 |
| 38.60 | 1.18 | 4.00 | 2.34 | 0.04 |

### Example 8

### Compound I/D-Tartaric Acid, -Malonic Acid, -L-Malic Acid, -D-Malic Acid Co-Crystal Forms

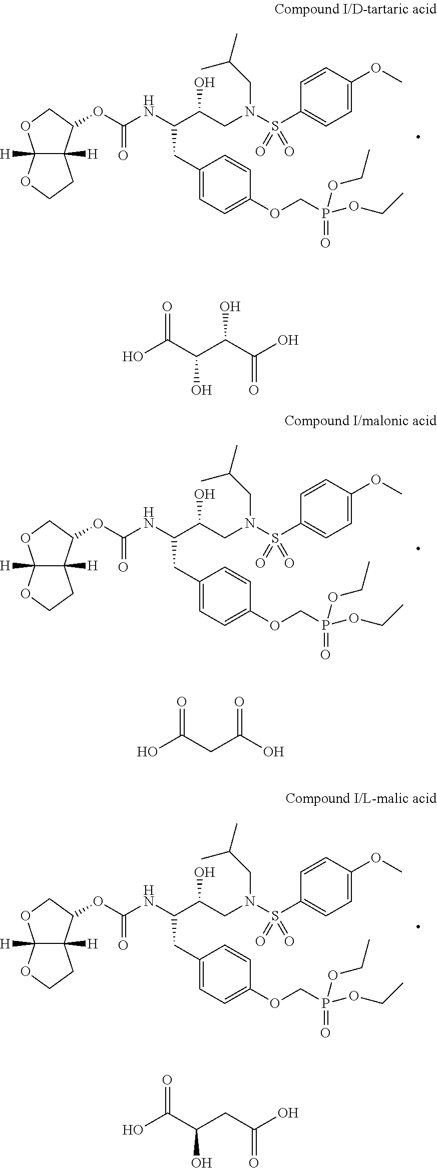

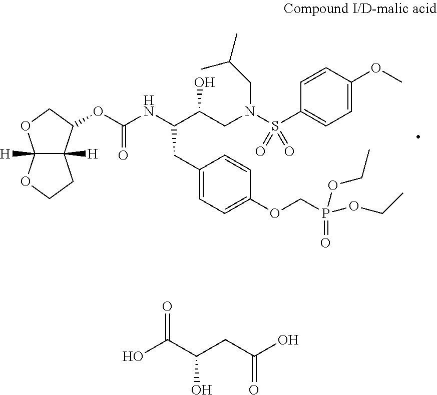

Compound I/D-tartaric acid, -malonic acid, -L-malic acid, -D-malic acid co-crystal forms are prepared by the following representative procedure (with malonic acid). 1.0 g Compound I (1.35 mmol) is combined with 140.5 mg malonic acid (1.35 mmol, 1.0 equiv.). The mixture is dissolved in THF (1.0 mL) and stirred overnight in a capped vial. The following day, the vial is vented and the solvent slowly evaporated over 4 days, producing a resin. On the fourth day, the hardened resin is seeded with 10.0 mg Compound I/L-tartaric acid co-crystal Form A (1.0% w/w). Upon standing overnight, the resins has crystallized into solids. The solids are analyzed by DSC and XRPD. Tables 5-8 shows (XRPD) peaks data of Compound I/D-tartaric acid, -malonic acid, -L-malic acid, -D-malic acid co-crystal forms.

TABLE 5
The XRPD Peak Data of Compound I/D-
tartaric acid Co-crystal Form

| | | FWHM | | |
|---|---|---|---|---|
| Pos.[°2Th.] | Height[cts] | [°2Th.] | d-spacing[Å] | Rel. Int.[%] |
| 4.19 | 2.60 | 4.00 | 21.05 | 0.83 |
| 4.20 | 1.30 | 4.00 | 21.05 | 0.42 |
| 5.41 | 235.21 | 0.30 | 16.32 | 75.05 |
| 5.42 | 117.61 | 0.30 | 16.32 | 37.52 |
| 7.78 | 48.57 | 0.21 | 11.35 | 15.50 |
| 7.80 | 24.29 | 0.21 | 11.35 | 7.75 |
| 10.63 | 248.47 | 0.20 | 8.31 | 79.28 |
| 10.66 | 124.23 | 0.20 | 8.31 | 39.64 |
| 11.38 | 52.12 | 0.21 | 7.77 | 16.63 |
| 11.41 | 26.06 | 0.21 | 7.77 | 8.31 |
| 12.51 | 124.56 | 0.11 | 7.07 | 39.74 |
| 12.54 | 62.28 | 0.11 | 7.07 | 19.87 |
| 12.83 | 226.05 | 0.15 | 6.89 | 72.12 |
| 12.86 | 113.02 | 0.15 | 6.89 | 36.06 |
| 14.70 | 71.07 | 0.23 | 6.02 | 22.68 |
| 14.74 | 35.53 | 0.23 | 6.02 | 11.34 |
| 15.16 | 204.69 | 0.14 | 5.84 | 65.31 |
| 15.20 | 102.34 | 0.14 | 5.84 | 32.65 |
| 15.36 | 139.31 | 0.14 | 5.77 | 44.45 |
| 15.40 | 69.65 | 0.14 | 5.77 | 22.22 |
| 15.79 | 134.55 | 0.15 | 5.61 | 42.93 |
| 15.83 | 67.27 | 0.15 | 5.61 | 21.46 |
| 17.50 | 36.81 | 0.17 | 5.06 | 11.74 |
| 17.55 | 18.40 | 0.17 | 5.06 | 5.87 |
| 17.91 | 64.05 | 0.18 | 4.95 | 20.44 |
| 17.95 | 32.03 | 0.18 | 4.95 | 10.22 |
| 19.09 | 313.42 | 0.19 | 4.65 | 100.00 |
| 19.14 | 156.71 | 0.19 | 4.65 | 50.00 |
| 19.37 | 233.83 | 0.28 | 4.58 | 74.60 |
| 19.42 | 116.91 | 0.28 | 4.58 | 37.30 |
| 20.12 | 44.11 | 0.16 | 4.41 | 14.07 |
| 20.17 | 22.06 | 0.16 | 4.41 | 7.04 |
| 20.61 | 137.73 | 0.18 | 4.31 | 43.94 |
| 20.66 | 68.86 | 0.18 | 4.31 | 21.97 |
| 21.21 | 16.91 | 0.12 | 4.19 | 5.40 |
| 21.26 | 8.46 | 0.12 | 4.19 | 2.70 |
| 22.31 | 87.70 | 0.08 | 3.98 | 27.98 |
| 22.37 | 43.85 | 0.08 | 3.98 | 13.99 |
| 22.63 | 70.12 | 0.27 | 3.93 | 22.37 |
| 22.69 | 35.06 | 0.27 | 3.93 | 11.19 |
| 23.72 | 155.86 | 0.19 | 3.75 | 49.73 |
| 23.78 | 77.93 | 0.19 | 3.75 | 24.86 |
| 24.31 | 176.12 | 0.16 | 3.66 | 56.19 |
| 24.37 | 88.06 | 0.16 | 3.66 | 28.10 |
| 25.00 | 78.59 | 0.27 | 3.56 | 25.07 |

TABLE 6
The XRPD Peak Data of Compound
I/malonic acid Co-crystal Form

| | | FWHM | | |
|---|---|---|---|---|
| Pos.[°2Th.] | Height[cts] | [°2Th.] | d-spacing[Å] | Rel. Int.[%] |
| 4.23 | 70.52 | 0.09 | 20.89 | 17.14 |
| 4.24 | 35.26 | 0.09 | 20.89 | 8.57 |
| 5.40 | 275.68 | 0.13 | 16.35 | 67.00 |
| 5.41 | 137.84 | 0.13 | 16.35 | 33.50 |
| 10.69 | 411.46 | 0.13 | 8.27 | 100.00 |
| 10.72 | 205.73 | 0.13 | 8.27 | 50.00 |
| 11.67 | 127.90 | 0.12 | 7.58 | 31.09 |
| 11.70 | 63.95 | 0.12 | 7.58 | 15.54 |
| 12.42 | 64.90 | 0.14 | 7.12 | 15.77 |
| 12.45 | 32.45 | 0.14 | 7.12 | 7.89 |
| 13.03 | 92.49 | 0.13 | 6.79 | 22.48 |
| 13.06 | 46.24 | 0.13 | 6.79 | 11.24 |
| 14.49 | 49.96 | 0.08 | 6.11 | 12.14 |
| 14.52 | 24.98 | 0.08 | 6.11 | 6.07 |
| 14.70 | 70.49 | 0.11 | 6.02 | 17.13 |
| 14.73 | 35.25 | 0.11 | 6.02 | 8.57 |
| 15.51 | 149.47 | 0.17 | 5.71 | 36.33 |
| 15.55 | 74.73 | 0.17 | 5.71 | 18.16 |
| 16.09 | 56.43 | 0.10 | 5.50 | 13.71 |
| 16.14 | 28.21 | 0.10 | 5.50 | 6.86 |
| 17.84 | 14.12 | 1.49 | 4.97 | 3.43 |
| 17.88 | 7.06 | 1.49 | 4.97 | 1.72 |
| 19.25 | 259.98 | 0.12 | 4.61 | 63.18 |
| 19.30 | 129.99 | 0.12 | 4.61 | 31.59 |
| 19.59 | 321.88 | 0.20 | 4.53 | 78.23 |
| 19.64 | 160.94 | 0.20 | 4.53 | 39.11 |
| 20.99 | 177.24 | 0.08 | 4.23 | 43.08 |
| 21.04 | 88.62 | 0.08 | 4.23 | 21.54 |
| 22.46 | 70.00 | 0.15 | 3.96 | 17.01 |
| 22.52 | 35.00 | 0.15 | 3.96 | 8.51 |
| 24.17 | 154.70 | 0.22 | 3.68 | 37.60 |
| 24.23 | 77.35 | 0.22 | 3.68 | 18.80 |
| 24.88 | 67.10 | 0.14 | 3.58 | 16.31 |
| 24.94 | 33.55 | 0.14 | 3.58 | 8.15 |
| 25.85 | 40.55 | 0.09 | 3.44 | 9.86 |
| 25.92 | 20.28 | 0.09 | 3.44 | 4.93 |
| 26.75 | 55.66 | 0.07 | 3.33 | 13.53 |
| 26.82 | 27.83 | 0.07 | 3.33 | 6.76 |
| 27.71 | 47.69 | 0.09 | 3.22 | 11.59 |
| 27.78 | 23.84 | 0.09 | 3.22 | 5.79 |
| 29.12 | 84.52 | 0.10 | 3.06 | 20.54 |
| 29.19 | 42.26 | 0.10 | 3.06 | 10.27 |
| 33.97 | 14.24 | 2.83 | 2.64 | 3.46 |
| 34.06 | 7.12 | 2.83 | 2.64 | 1.73 |

TABLE 7
The XRPD Peak Data of Compound
I/L-malic acid Co-crystal Form

| | | FWHM | | |
|---|---|---|---|---|
| Pos.[°2Th.] | Height[cts] | [°2Th.] | d-spacing[Å] | Rel. Int.[%] |
| 4.11 | 148.60 | 0.10 | 21.47 | 24.33 |
| 4.12 | 74.30 | 0.10 | 21.47 | 12.17 |
| 5.24 | 480.74 | 0.12 | 16.85 | 78.73 |
| 5.25 | 240.37 | 0.12 | 16.85 | 39.36 |
| 7.70 | 70.65 | 0.13 | 11.47 | 11.57 |
| 7.72 | 35.33 | 0.13 | 11.47 | 5.79 |
| 10.46 | 382.20 | 0.10 | 8.45 | 62.59 |
| 10.49 | 191.10 | 0.10 | 8.45 | 31.29 |
| 11.34 | 67.25 | 0.21 | 7.79 | 11.01 |
| 11.37 | 33.62 | 0.21 | 7.79 | 5.51 |
| 12.45 | 157.49 | 0.10 | 7.11 | 25.79 |
| 12.48 | 78.75 | 0.10 | 7.11 | 12.90 |
| 12.69 | 242.03 | 0.09 | 6.97 | 39.64 |
| 12.73 | 121.02 | 0.09 | 6.97 | 19.82 |
| 14.46 | 131.41 | 0.14 | 6.12 | 21.52 |
| 14.49 | 65.70 | 0.14 | 6.12 | 10.76 |
| 15.19 | 181.03 | 0.12 | 5.83 | 29.65 |
| 15.23 | 90.51 | 0.12 | 5.83 | 14.82 |
| 15.41 | 180.26 | 0.08 | 5.75 | 29.52 |
| 15.45 | 90.13 | 0.08 | 5.75 | 14.76 |
| 15.72 | 110.78 | 0.06 | 5.63 | 18.14 |
| 15.76 | 55.39 | 0.06 | 5.63 | 9.07 |
| 18.09 | 9.39 | 4.00 | 4.90 | 1.54 |
| 18.13 | 4.70 | 4.00 | 4.90 | 0.77 |
| 19.14 | 610.65 | 0.10 | 4.63 | 100.00 |
| 19.19 | 305.33 | 0.10 | 4.63 | 50.00 |
| 20.09 | 59.40 | 0.07 | 4.42 | 9.73 |
| 20.14 | 29.70 | 0.07 | 4.42 | 4.86 |
| 20.52 | 79.29 | 0.19 | 4.32 | 12.99 |
| 20.57 | 39.65 | 0.19 | 4.32 | 6.49 |
| 22.49 | 98.09 | 0.13 | 3.95 | 16.06 |
| 22.55 | 49.04 | 0.13 | 3.95 | 8.03 |
| 23.65 | 119.19 | 0.10 | 3.76 | 19.52 |
| 23.71 | 59.59 | 0.10 | 3.76 | 9.76 |
| 24.04 | 163.93 | 0.11 | 3.70 | 26.84 |
| 24.10 | 81.96 | 0.11 | 3.70 | 13.42 |
| 25.02 | 107.45 | 0.13 | 3.56 | 17.60 |
| 25.08 | 53.73 | 0.13 | 3.56 | 8.80 |
| 29.14 | 21.45 | 0.22 | 3.06 | 3.51 |
| 29.21 | 10.73 | 0.22 | 3.06 | 1.76 |
| 33.05 | 42.40 | 0.09 | 2.71 | 6.94 |
| 33.14 | 21.20 | 0.09 | 2.71 | 3.47 |
| 35.19 | 15.97 | 0.12 | 2.55 | 2.61 |
| 35.28 | 7.98 | 0.12 | 2.55 | 1.31 |

TABLE 8
The XRPD Peak Data of Compound
I/D-malic acid Co-crystal Form

| | | FWHM | | |
|---|---|---|---|---|
| Pos.[°2Th.] | Height[cts] | [°2Th.] | d-spacing[Å] | Rel. Int.[%] |
| 4.09 | 140.81 | 0.13 | 21.60 | 16.63 |
| 4.10 | 70.40 | 0.13 | 21.60 | 8.32 |
| 5.26 | 776.88 | 0.10 | 16.77 | 91.77 |
| 5.28 | 388.44 | 0.10 | 16.77 | 45.89 |
| 7.64 | 77.00 | 0.08 | 11.56 | 9.10 |
| 7.66 | 38.50 | 0.08 | 11.56 | 4.55 |
| 10.50 | 846.53 | 0.08 | 8.41 | 100.00 |
| 10.53 | 423.26 | 0.08 | 8.41 | 50.00 |
| 11.11 | 78.00 | 0.08 | 7.96 | 9.21 |
| 11.14 | 39.00 | 0.08 | 7.96 | 4.61 |
| 11.37 | 83.52 | 0.08 | 7.78 | 9.87 |
| 11.40 | 41.76 | 0.08 | 7.78 | 4.93 |
| 12.37 | 164.44 | 0.08 | 7.15 | 19.43 |
| 12.40 | 82.22 | 0.08 | 7.15 | 9.71 |
| 12.74 | 227.83 | 0.07 | 6.94 | 26.91 |
| 12.78 | 113.91 | 0.07 | 6.94 | 13.46 |
| 14.44 | 101.09 | 0.07 | 6.13 | 11.94 |
| 14.48 | 50.54 | 0.07 | 6.13 | 5.97 |
| 14.56 | 110.05 | 0.07 | 6.08 | 13.00 |
| 14.60 | 55.03 | 0.07 | 6.08 | 6.50 |
| 14.90 | 69.96 | 0.11 | 5.94 | 8.26 |
| 14.93 | 34.98 | 0.11 | 5.94 | 4.13 |
| 15.17 | 132.44 | 0.07 | 5.83 | 15.64 |
| 15.21 | 66.22 | 0.07 | 5.83 | 7.82 |
| 15.29 | 148.97 | 0.10 | 5.79 | 17.60 |
| 15.33 | 74.48 | 0.10 | 5.79 | 8.80 |
| 15.77 | 90.11 | 0.05 | 5.62 | 10.64 |
| 15.81 | 45.05 | 0.05 | 5.62 | 5.32 |
| 17.35 | 64.51 | 0.01 | 5.11 | 7.62 |
| 17.39 | 32.25 | 0.01 | 5.11 | 3.81 |
| 18.52 | 9.03 | 3.40 | 4.79 | 1.07 |
| 18.56 | 4.51 | 3.40 | 4.79 | 0.53 |
| 19.17 | 609.24 | 0.09 | 4.63 | 71.97 |
| 19.22 | 304.62 | 0.09 | 4.63 | 35.98 |
| 20.21 | 8.67 | 1.70 | 4.39 | 1.02 |
| 20.27 | 4.34 | 1.70 | 4.39 | 0.51 |
| 20.58 | 100.28 | 0.09 | 4.31 | 11.85 |
| 20.64 | 50.14 | 0.09 | 4.31 | 5.92 |
| 22.31 | 73.64 | 0.10 | 3.98 | 8.70 |
| 22.36 | 36.82 | 0.10 | 3.98 | 4.35 |
| 23.70 | 110.96 | 0.06 | 3.75 | 13.11 |
| 23.76 | 55.48 | 0.06 | 3.75 | 6.55 |
| 24.06 | 122.18 | 0.14 | 3.70 | 14.43 |
| 24.12 | 61.09 | 0.14 | 3.70 | 7.22 |
| 24.87 | 92.36 | 0.11 | 3.58 | 10.91 |
| 24.94 | 46.18 | 0.11 | 3.58 | 5.46 |
| 26.41 | 68.55 | 0.06 | 3.37 | 8.10 |
| 26.48 | 34.27 | 0.06 | 3.37 | 4.05 |
| 29.37 | 37.61 | 0.10 | 3.04 | 4.44 |
| 29.44 | 18.80 | 0.10 | 3.04 | 2.22 |
| 30.01 | 8.58 | 0.85 | 2.98 | 1.01 |
| 30.09 | 4.29 | 0.85 | 2.98 | 0.51 |
| 32.91 | 49.00 | 0.08 | 2.72 | 5.79 |
| 32.99 | 24.50 | 0.08 | 2.72 | 2.89 |
| 33.89 | 0.35 | 0.00 | 2.64 | 0.04 |
| 33.98 | 0.17 | 0.00 | 2.64 | 0.02 |
| 35.18 | 47.19 | 0.04 | 2.55 | 5.57 |
| 35.27 | 23.59 | 0.04 | 2.55 | 2.79 |

### Example 9

### Compound I/Benzoic Acid Co-Crystal Form

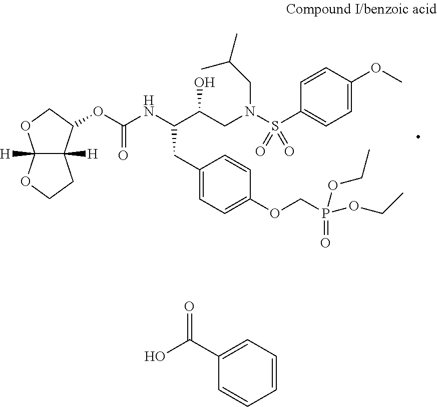

A solution of Compound I and benzoic acid in THF produced crystalline solid over several weeks from an initial clear, colorless resin.

A 20 mL glass reactor was charged with 810 mg Compound I and 2.0 mL THF. A 1-dram threaded glass vial was charged with 140 mg benzoic acid (Sigma, 07905LH) and 2.0 mL THF.

Both solids fully dissolved without delay at 21° C. The benzoic acid solution was quantitatively added to the Compound I solution using a 1.0 mL THF wash. The resulting solution was clear and colorless, and was allowed to evaporate unassisted at 21° C. with constant stirring (stir bar).

After ˜24 hours, the solution became a rigid, clear, colorless resin and the stir bar was immobilized. Examination with a 14× magnification monocular showed no sign of solids. The open vial was dried under vacuum at 21° C.

After ˜48 hours under vacuum, a 14× monocular examination of the material revealed no changes. The resin and walls were scratched with a metal spatula to induce crystallization and the sample was set in a fume hood to age at 21° C.

Over the course of 3 to 4 months the status of the solution was examined several times with the naked eye and with a 14× monocular eyepiece. During this period, a slow progression from clear colorless resin to a solid mass of densely packed very fine needle-like crystals was observed. When the transformation appeared complete, a XRPD was obtained and other characterization steps were done.

Table 9 shows (XRPD) peaks data of Compound I/benzoic acid co-crystal form.

TABLE 9
The XRPD Peak Data of Compound
I/benzoic acid Co-crystal Form

| | | FWHM | | |
|---|---|---|---|---|
| Pos.[°2Th.] | Height[cts] | [°2Th.] | d-spacing[Å] | Rel. Int.[%] |
| 4.51 | 82.63 | 0.17 | 19.57 | 41.88 |
| 4.52 | 41.32 | 0.17 | 19.57 | 20.94 |
| 6.24 | 57.49 | 0.15 | 14.14 | 29.14 |
| 6.26 | 28.75 | 0.15 | 14.14 | 14.57 |
| 7.19 | 23.45 | 0.13 | 12.29 | 11.89 |
| 7.21 | 11.73 | 0.13 | 12.29 | 5.94 |
| 9.85 | 151.63 | 0.14 | 8.97 | 76.85 |
| 9.88 | 75.82 | 0.14 | 8.97 | 38.42 |
| 10.09 | 11.33 | 4.00 | 8.76 | 5.74 |
| 10.12 | 5.67 | 4.00 | 8.76 | 2.87 |
| 11.34 | 34.72 | 0.19 | 7.80 | 17.59 |
| 11.37 | 17.36 | 0.19 | 7.80 | 8.80 |
| 12.39 | 82.00 | 0.14 | 7.14 | 41.56 |
| 12.42 | 41.00 | 0.14 | 7.14 | 20.78 |
| 12.81 | 94.95 | 0.15 | 6.90 | 48.12 |
| 12.84 | 47.47 | 0.15 | 6.90 | 24.06 |
| 13.22 | 28.29 | 0.15 | 6.69 | 14.34 |
| 13.25 | 14.15 | 0.15 | 6.69 | 7.17 |
| 14.25 | 34.11 | 0.36 | 6.21 | 17.29 |
| 14.29 | 17.05 | 0.36 | 6.21 | 8.64 |
| 16.34 | 91.64 | 0.12 | 5.42 | 46.44 |
| 16.38 | 45.82 | 0.12 | 5.42 | 23.22 |
| 16.75 | 122.68 | 0.12 | 5.29 | 62.17 |
| 16.79 | 61.34 | 0.12 | 5.29 | 31.09 |
| 17.01 | 105.56 | 0.25 | 5.21 | 53.50 |
| 17.05 | 52.78 | 0.25 | 5.21 | 26.75 |
| 17.86 | 85.10 | 0.16 | 4.96 | 43.13 |
| 17.91 | 42.55 | 0.16 | 4.96 | 21.56 |
| 18.13 | 105.48 | 0.14 | 4.89 | 53.46 |
| 18.18 | 52.74 | 0.14 | 4.89 | 26.73 |
| 18.58 | 34.24 | 0.07 | 4.77 | 17.35 |
| 18.63 | 17.12 | 0.07 | 4.77 | 8.68 |
| 18.95 | 58.61 | 0.18 | 4.68 | 29.70 |
| 19.00 | 29.31 | 0.18 | 4.68 | 14.85 |
| 19.59 | 126.24 | 0.27 | 4.53 | 63.98 |
| 19.64 | 63.12 | 0.27 | 4.53 | 31.99 |
| 20.20 | 197.31 | 0.24 | 4.39 | 100.00 |
| 20.25 | 98.66 | 0.24 | 4.39 | 50.00 |
| 20.85 | 33.69 | 0.15 | 4.26 | 17.07 |
| 20.90 | 16.84 | 0.15 | 4.26 | 8.54 |
| 21.40 | 89.87 | 0.12 | 4.15 | 45.55 |
| 21.46 | 44.93 | 0.12 | 4.15 | 22.77 |
| 21.87 | 57.23 | 0.19 | 4.06 | 29.00 |
| 21.92 | 28.61 | 0.19 | 4.06 | 14.50 |
| 22.31 | 54.36 | 0.12 | 3.98 | 27.55 |
| 22.37 | 27.18 | 0.12 | 3.98 | 13.77 |
| 23.20 | 91.20 | 0.40 | 3.83 | 46.22 |
| 23.25 | 45.60 | 0.40 | 3.83 | 23.11 |
| 24.74 | 46.18 | 0.31 | 3.60 | 23.40 |
| 24.80 | 23.09 | 0.31 | 3.60 | 11.70 |
| 25.04 | 50.50 | 0.13 | 3.55 | 25.60 |
| 25.10 | 25.25 | 0.13 | 3.55 | 12.80 |
| 25.74 | 46.27 | 0.19 | 3.46 | 23.45 |
| 25.81 | 23.13 | 0.19 | 3.46 | 11.72 |
| 26.33 | 31.33 | 0.91 | 3.38 | 15.88 |
| 26.39 | 15.66 | 0.91 | 3.38 | 7.94 |
| 28.23 | 66.52 | 0.24 | 3.16 | 33.71 |
| 28.30 | 33.26 | 0.24 | 3.16 | 16.86 |
| 29.78 | 12.16 | 1.41 | 3.00 | 6.16 |
| 29.86 | 6.08 | 1.41 | 3.00 | 3.08 |
| 30.80 | 22.42 | 0.20 | 2.90 | 11.36 |
| 30.88 | 11.21 | 0.20 | 2.90 | 5.68 |
| 32.23 | 13.69 | 0.16 | 2.78 | 6.94 |
| 32.31 | 6.84 | 0.16 | 2.78 | 3.47 |
| 32.75 | 17.27 | 0.15 | 2.73 | 8.75 |
| 32.83 | 8.64 | 0.15 | 2.73 | 4.38 |
| 34.20 | 3.40 | 2.12 | 2.62 | 1.72 |
| 34.29 | 1.70 | 2.12 | 2.62 | 0.86 |

1. A crystalline form of (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate having a X-ray powder diffraction pattern comprising characteristic peaks at diffraction angles expressed in degrees 2-theta of about 4.9, 6.4, 9.8, 9.8, 10.4, 10.5, 13.6, 14.7, 15.6, 17.6, 18.3, and 24.7.

2. The crystalline form of claim 1, wherein the polymorph is a substantially pure polymorph.

3. A crystalline form of (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate having substantially the same X-ray powder diffraction pattern shown in FIG. 1.

4. The crystalline form of claim 3, wherein the polymorph is a substantially pure polymorph.

5. The crystalline form of any of claim 1, wherein the polymorph has a DSC extrapolated melting temperature onset of about 54° C.

6. The crystalline form of claim 1, wherein said polymorph comprises about 1.4% to about 2.4% by weight of water.

7. A co-crystal comprising (3R,3aS,6aR)-hexahydrofuro[2,3-b]furan-3-yl (1S,2R)-(1-{4-[(diethoxyphosphoryl)methoxy]phenyl}-3-hydroxy-4-[4-methoxy-N-(2-methylpropyl)benzenesulfonamido]butan-2-yl)carbamate and a co-crystal former.

8. The co-crystal of claim 7, said co-crystal former being selected from the group consisting of L-tartaric acid, D-tartaric acid, malonic acid, L-malic acid, D-malic acid and benzoic acid.

9. The co-crystal of claim 8, wherein the co-crystal former is L-tartaric acid.

10. The co-crystal Form A of claim 9 having a X-ray powder diffraction pattern comprising characteristic peaks at diffraction angles expressed in degrees 2-theta of about 5.9, 7.9, 11.1, 11.6, 13.3, 13.9, 15.7, 16.8 and 17.9.

11. The co-crystal Form A of claim 9 having substantially the same X-ray diffraction pattern shown in FIG. 7.

12. The co-crystal Form B of claim 9 having a X-ray powder diffraction pattern comprising characteristic peaks at diffraction angles expressed in degrees 2-theta of about 4.1, 5.3, 7.7, 9.6, 10.6, 11.3, 12.4, 15.1 15.7, 19.1, and 19.3.

13. The co-crystal Form B of claim 9 having substantially the same X-ray diffraction pattern shown in FIG. 11.

14. The co-crystal of claim 8, wherein the co-crystal former is D-tartaric acid.

15. The co-crystal of claim 14 having a X-ray powder diffraction pattern comprising characteristic peaks at diffraction angles expressed in degrees 2-theta of about 4.1, 5.3, 7.7, 7.9, 10.6, 11.4, 12.5, 12.8, 14.6 and 15.2.

16. The co-crystal of claim 14 having substantially the same X-ray diffraction pattern shown in FIG. 22.

17. The co-crystal of claim 8, wherein the co-crystal former is malonic acid.

18. The co-crystal of claim 17 having a X-ray powder diffraction pattern comprising characteristic peaks at diffraction angles expressed in degrees 2-theta of about 4.2, 5.4, 10.7, 11.6, 12.4, 13.0, 15.4, 16.1 and 19.5.

19. The co-crystal of claim 17 having substantially the same X-ray diffraction pattern shown in FIG. 14.

20. The co-crystal of claim 8, wherein the co-crystal former is L-malic acid.

21. The co-crystal of claim 20 having a X-ray powder diffraction pattern comprising characteristic peaks at diffraction angles expressed in degrees 2-theta of about 4.1, 5.3, 7.7, 10.5, 11.4, 12.7, 14.5, 15.2 and 19.2.

22. The co-crystal of claim 20 having substantially the same X-ray diffraction pattern shown in FIG. 16.

23. The co-crystal of claim 8, wherein the co-crystal former is D-malic acid.

24. The co-crystal of claim 23 having a X-ray powder diffraction pattern comprising characteristic peaks at diffraction angles expressed in degrees 2-theta of about 4.1, 5.3, 7.6, 10.5, 11.1, 12.4, 12.7, 14.6, 15.5 and 19.2.

25. The co-crystal of claim 23 having substantially the same X-ray diffraction pattern shown in FIG. 18.

26. The co-crystal of claim 8, wherein the co-crystal former is benzoic acid.

27. The co-crystal of claim 26 having a X-ray powder diffraction pattern comprising characteristic peaks at diffraction angles expressed in degrees 2-theta of about 4.5, 6.3, 7.2, 9.9, 11.4, 12.4, 12.8, 14.3, 16.4 and 16.8

28. The co-crystal of claim 27 having substantially the same X-ray diffraction pattern shown in FIG. 20.

29. A pharmaceutical composition comprising a crystalline form according to claim 1 and a pharmaceutically acceptable carrier.

30. A pharmaceutical composition consisting essentially of the crystalline form according to claim 1 and a pharmaceutically acceptable carrier.

31. A method of inhibiting activity of a HIV protease in a subject comprising administering to the subject a therapeutically effective amount of the crystalline form according to claim 1.

32. A method of treating HIV infection or conditions associated with HIV infection comprising administering to the subject a therapeutically effective amount of the crystalline form according to claim 1.

33-34. (canceled)

35. A pharmaceutical composition consisting essentially of the crystalline form according to claim 7 and a pharmaceutically acceptable carrier.

36. A method of inhibiting activity of a HIV protease in a subject comprising administering to the subject a therapeutically effective amount of the crystalline form according to claim 7.

37. A method of treating HIV infection or conditions associated with HIV infection comprising administering to the subject a therapeutically effective amount of the crystalline form according to claim 7.
